(19) United States
(12) Patent Application Publication
(10) Pub. No.: US 2024/0043936 A1
(43) Pub. Date: Feb. 8, 2024

(54) PROGNOSTIC BIOMARKERS FOR BREAST CANCER

(71) Applicant: Sanford Burnham Prebys Medical Discovery Institute, La Jolla, CA (US)

(72) Inventor: Svasti HARICHARAN, San Diego, CA (US)

(21) Appl. No.: 18/251,045

(22) PCT Filed: Oct. 27, 2021
(86) PCT No.: PCT/US21/56808

§ 371 Date: Apr. 28, 2023

Related U.S. Application Data
(60) Provisional application No. 63/106,777, filed on Oct. 28, 2020.

(51) Int. Cl. C12Q 1/6886; A61K 45/06

(52) U.S. Cl. CPC C12Q 1/6886; A61K 45/06; C12Q 2600/118; C12Q 2600/158

(57) ABSTRACT

Described herein are methods of prognosing/diagnosing an estrogen receptor positive (ER⁺) breast cancer in an African American subject. Also described herein are methods of treating an estrogen receptor positive (ER⁺) breast cancer in an African American subject.

### CROSS REFERENCE TO RELATED APPLICATIONS

This application claims the benefit of U.S. Provisional Patent Application No. 63/106,777 filed on Oct. 28, 2020. Priority is claimed pursuant to 35 U.S.C. § 119. The above noted patent application is incorporated by reference as if set forth fully herein.

### STATEMENT REGARDING FEDERALLY SPONSORED RESEARCH

This invention was made with government support under K22 CA229613 awarded by the National Institutes of Health and W81XWH-18-1-0034 awarded by the Department of Defense. The government has certain rights in the invention.

### INCORPORATION BY REFERENCE

All publications, patents, and patent applications mentioned in this specification are herein incorporated by reference, in their entireties, to the same extent as if each individual publication, patent, or patent application was specifically and individually indicated to be incorporated by reference.

### BACKGROUND

African Americans (AAs) constitute a racial group with the highest mortality rate across cancer types. In AA women, breast, lung and colorectal are the three most common cancer diagnoses (see Furberg H et al., Breast Cancer Res Treat. 2001; 68(1):33-43; Gao R et al., Mol Cancer Ther. 2008; 7(5):1246-1250; Haddad S A et al., Breast Cancer Res Treat. 2015; 154(1):145-154; Breast Cancer Rates Among Black Women and White Women CDC. Published Jan. 31, 2019. Accessed Oct. 20, 2020). Breast cancer accounts for 32% of these diagnoses, making it one of the most predominant cancer types in AA women, as it is in Caucasian Americans (CAs) (see Breast Cancer Rates Among Black Women and White Women CDC. Published Jan. 31, 2019. Accessed Oct. 20, 2020). Although AA women typically have higher incidence of triple negative breast cancer than CAs, estrogen receptor positive (ER*) breast cancer remains the most commonly diagnosed subtype of breast cancer in AA women, as it is in CAs (see Gao R et al., Mol Cancer Ther. 2008; 7(5):1246-1250; Haddad S A et al., Breast Cancer Res Treat. 2015; 154(1):145-154; Breast Cancer Rates Among Black Women and White Women| CDC. Published Jan. 31, 2019. Accessed Oct. 20, 2020). AA women with ER⁺ breast cancer present with higher tumor grade and more advanced disease that is more frequently luminal B, and therefore, resistant to standard endocrine therapies than CAs. AA ER⁺ breast cancer patients are more likely to have poor clinical outcome with 42% higher mortality rate than CAs (see Rauscher G H et al., Breast Cancer Res Treat. 2017; 163(2):321-330; Menashe I et al., NCI J Natl Cancer Inst. 2009; 101(14):993-1000; Ma H et al., MC Cancer. 2013; 13:225; Ren Y et al., PLOS ONE. 2014; 9(10):e110281; Menendez-Tuckman A T et al., Natl Cancer Inst. 1994; 86(17):1352-1353).

There is a consensus in the literature that environmental/socioeconomic factors contribute to poor breast cancer outcome in AAs (see Rauscher G H et al., Breast Cancer Res Treat. 2017; 163(2):321-330; Menashe I et al., NCI J Natl Cancer Inst. 2009; 101(14):993-1000; Rauscher G H et al., Am J Epidemiol. 2016; 183(10):884-893; Warner E T et al., J Clin Oncol Off J Am Soc Clin Oncol. 2015; 33(20):2254-2261). These include reproductive factors and socioeconomic factors including access to healthcare (see Costantino N S et al., Ethn Dis. 26(3):407-416; Lovejoy L A et al., Ann Surg Oncol. 2019; 26(12):3838-3845; Watlington A T et al., Cancer. 2007; 109(10):2093-2099; Wojcik B E et al., Breast J. 2003; 9(3):175-183; Bao P-P et al., Cancer Causes Control CCC. 2016; 27(2):229-236; Torio C M et al., m J Pubic Health. 2010; 100(1):146-151). However, even when these factors are controlled for, differences in presentation and outcome persist in AA ER⁺ breast cancer patients (see Wojcik B E et al., Breast J. 2003; 9(3):175-183). Therefore, it indicates that ER⁺ tumor formation and progression in AA patients has a distinct molecular profile, as suggested by previous studies (see Troester M A et al., J Natl Cancer Inst. 2018; 110(2); Comparison of Breast Cancer Molecular Features and Survival by African and European Ancestry in The Cancer Genome Atlas| Breast Cancer| JAMA Oncology|JAMA Network; Accessed Oct. 20, 2020; Sparano J A et al., JNCI J Natl Cancer Inst. 2012; 104(5):406-414). Understanding this profile is critical for developing precision medicine approaches tailored to this underserved population. Such efforts require comprehensive and race-specific molecular characterization of tumors.

The methods described herein stem from such efforts in understanding the unique molecular profile of AA breast cancer patients, and satisfy the need of race-tailored the medical care and provide related advantages.

### SUMMARY

Disclosed herein, in one aspect, is a method of prognosing and/or diagnosing an estrogen receptor positive (ER⁺) breast cancer in an African American subject in need thereof, comprising a. measuring a level of expression of one or more DNA damage repair (DDR) genes in a sample from the African American subject, wherein the one or more DDR genes is selected from the group consisting of XPC, XPA, RBX1, RAD23B, RAD23A, MNAT1, GTF2H5, GTF2H4, GTF2H3, GTF2H2, GTF2H1, ERCC8, ERCC6, ERCC5, ERCC4, ERCC3, ERCC2, ERCC1, DDB2, DDB1, CUL4B, CUL4A, CETN2, XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, APEX1, PMS2P3, PMS2, PMS1, MSH6, MSH5, MSH4, MSH3, MSH2, MLH3, MLH1, EXO1, XRCC6, XRCC5, XRCC4, PRKDC, POLM, NHEJ1, LIG4, DNTT, XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, BLM, SLX4, PALB2, FANCM, FANCL, FANCI, FANCG, FANCF, FANCE, FANCD2, FANC, FANCB, FANCA, FAAP24, FAAP20, and BRIP1; and b. comparing the level of expression to a control level of expression of the one or more DDR genes to obtain a comparison expression value, thereby prognosing and/or diagnosing the ER+ breast cancer in the African American subject in need thereof. In some embodiments, the one or more of DDR genes comprise DDR genes encoding cell cycle checkpoint scaffold proteins. In some embodiments, the level of expression is up- or down-regulated compared to the control level of expression.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

In some cases, the method described herein further comprises prognosing the African American subject in need thereof as later having poor disease-specific survival rate, relapse-free survival rate, and/or overall survival rate.

In some cases, the method described herein further comprises administering to the African American subject in need thereof an effective amount of cyclin-dependent kinase inhibitor, endocrine therapy, surgery, chemotherapy, and/or radiation. In some specific cases, the cyclin-dependent kinase inhibitor comprises one or more inhibitors of cyclin-dependent kinase 1 (CDK-1), CDK-4, and CDK-6

In some cases, the African American subject in need thereof has a stage I, II, III, or IV breast cancer. In other cases, the African American subject in need thereof has a node status of 0, 1, 2, 3, or 3+. In other cases, the African American subject in need thereof has a breast cancer that has tested positive or negative for progesterone receptors. In other cases, the African American subject in need thereof has a breast cancer that has tested positive or negative for HER2 protein.

Disclosed herein, in another aspect, is a method of prognosing and/or diagnosing an estrogen receptor positive (ER⁺) breast cancer in an African American subject in need thereof, comprising a. measuring a level of expression of one or more of NBN, BRCA1, FANCE, MNAT1, NEIL3, XRCC4, XRCC1, and PARP1 in a sample from the African American subject; and b. comparing the level of expression to a control level of expression to obtain a comparison expression value, thereby prognosing and/or diagnosing the ER⁺ breast cancer in the African American subject in need thereof.

In some cases, the level of expression of one or more of NBN, BRCA1, FANCE, MNAT1, and NEIL3 is higher than the control level of expression. In some cases, the level of expression of one or more of XRCC4, XRCC1, and PARP1 is lower than the control level of expression.

In some cases, the method described herein further comprises measuring a level of expression of at least two or more of NBN, BRCA1, FANCE, MNAT1, NEIL3, XRCC4, XRCC1, and PARP1. In other cases, the method described herein further comprises measuring a level of expression of at least three or more of NBN, BRCA1, FANCE, MNAT1, NEIL3, XRCC4, XRCC1, and PARP1. In other cases, the method described herein further comprises measuring a level of expression of at least four or more of NBN, BRCA1, FANCE, MNAT1, NEIL3, XRCC4, XRCC1, and PARP1. In other cases, the method described herein further comprises measuring a level of expression of at least five or more of NBN, BRCA1, FANCE, MNAT1, NEIL3, XRCC4, XRCC1, and PARP1. In other cases, the method described herein further comprises measuring a level of expression of at least six or more of NBN, BRCA1, FANCE, MNAT1, NEIL3, XRCC4, XRCC1, and PARP1. In other cases, the method described herein further comprises measuring a level of expression of at least seven or more of NBN, BRCA1, FANCE, MNAT1, NEIL3, XRCC4, XRCC1, and PARP1. In other cases, the method described herein further comprises measuring a level of expression of NBN, BRCA1, FANCE, MNAT1, NEIL3, XRCC4, XRCC1, and PARP1.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

In some cases, the method described herein further comprises prognosing the African American subject in need thereof as later having poor disease-specific survival rate, relapse-free survival rate, and/or overall survival rate.

In some cases, the method described herein further comprises administering to the African American subject in need thereof an effective amount of cyclin-dependent kinase inhibitor, endocrine therapy, surgery, chemotherapy, and/or radiation.

In some cases, the African American subject in need thereof has a stage I, II, III, or IV breast cancer. In other cases, the African American subject in need thereof has a node status of 0, 1, 2, 3, or 3+. In other cases, the African American subject in need thereof has a breast cancer that has tested positive or negative for progesterone receptors. In other cases, the African American subject in need thereof has a breast cancer that has tested positive or negative for HER2 protein.

Disclosed herein, in another aspect, is a method of prognosing and/or diagnosing an estrogen receptor positive (ER⁺) breast cancer in an African American subject in need thereof, comprising identifying mutation(s) in one or more of BRCA1, FAAP24, FANCM, PARP4, PARP1, and ERCC6, thereby prognosing and/or diagnosing the ER⁺ breast cancer in the African American subject in need thereof. In some cases, the method described herein further comprises obtaining full or partial sequencing information of one or more of BRCA1, FAAP24, FANCM, PARP4, PARP1, and ERCC6.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the method described herein further comprises prognosing the African American subject in need thereof as later having poor disease-specific survival rate, relapse-free survival rate, and/or overall survival rate.

In some cases, the method described herein further comprises administering to the African American subject in need thereof an effective amount of cyclin-dependent kinase inhibitor, endocrine therapy, surgery, chemotherapy, and/or radiation.

In some cases, the African American subject in need thereof has a stage I, II, III, or IV breast cancer. In other cases, the African American subject in need thereof has a node status of 0, 1, 2, 3, or 3+. In other cases, the African American subject in need thereof has a breast cancer that has tested positive or negative for progesterone receptors. In other cases, the African American subject in need thereof has a breast cancer that has tested positive or negative for HER2 protein.

Disclosed herein, in another aspect, is a method of treating an estrogen receptor positive (ER⁺) breast cancer in a susceptible African American subject, comprising a. identifying the susceptible African American subject by i. measuring a level of expression of one or more of DNA damage repair (DDR) genes involved in a homologous recombination (HR) pathway, wherein the one or more DDR genes involved in the HR pathway are selected from the group consisting of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, and BLM; and a level of expression of one or more of DDR genes involved in a base excision repair (BER) pathway, wherein the one or more DDR genes involved in the BER pathway are selected from the group consisting of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1; and ii. comparing the level of expression to a control level of expression to obtain a comparison expression value; and b. administering to the susceptible African American subject an effective amount of one or more cyclin-dependent kinase 2 (CDK2) inhibitors, thereby treating the ER+ breast cancer in the African American subject. In some cases, the level of expression of one or more of DDR genes involved in the HR pathway is higher than the control level of expression, and the level of expression of one or more of DDR genes involved in the BER pathway is lower than the control level of expression in the susceptible African American subject.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Förster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

In some cases, the method described herein further comprises prognosing the African American subject in need thereof as later having poor disease-specific survival rate, relapse-free survival rate, and/or overall survival rate.

In some cases, the one or more CDK2 inhibitors comprise AT7519, AG-024322, Dinaciclib, Cyc065, Roniciclib, TG02, and/or Milciclib. In some cases, the method described herein further comprises administering to the African American subject in need thereof an effective amount of cyclin-dependent kinase inhibitor, endocrine therapy, surgery, chemotherapy, and/or radiation. In some cases, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

Disclosed herein, in another aspect, is a method of treating an estrogen receptor positive (ER+) breast cancer in a susceptible African American subject, comprising: a. identifying the susceptible African American subject by i. measuring a level of expression of one or more of DNA damage repair (DDR) genes involved in a homologous recombination (HR) pathway, wherein the one or more DDR genes involved in the HR pathway are selected from the group consisting of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, and BLM; and a level of expression of one or more of DDR genes involved in a base excision repair (BER) pathway, wherein the one or more DDR genes involved in the BER pathway are selected from the group consisting of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1; and ii. comparing the level of expression to a control level of expression to obtain a comparison expression value; and b. administering to the susceptible African American subject an effective amount of one or more proliferating cell nuclear antigen (PCNA) inhibitors, thereby treating the ER⁺ breast cancer in the African American subject. In some cases, the level of expression of one or more of DDR genes involved in the HR pathway is higher than the control level of expression, and the level of expression of one or more of DDR genes involved in the BER pathway is lower than the control level of expression in the susceptible African American subject.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

In some cases, the method described herein further comprises prognosing the African American subject in need thereof as later having poor disease-specific survival rate, relapse-free survival rate, and/or overall survival rate.

In some cases, the method described herein further comprises administering to the African American subject in need thereof an effective amount of cyclin-dependent kinase inhibitor, endocrine therapy, surgery, chemotherapy, and/or radiation. In some cases, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

Disclosed herein, in another aspect, is a method of treating an estrogen receptor positive (ER⁺) breast cancer in an African American subject in need thereof comprising administering an effective amount of one or more inhibitors of cyclin-dependent kinase 1 (CDK-1), CDK-4, and CDK-6.

In some cases, the one or more inhibitors of CDK-1, CDK-4, and CDK-6 comprise abemaciclib, Palbociclib, ribociclib, or a combination thereof. In some cases, the method described herein further comprises administering to the susceptible African American subject endocrine therapy, surgery, chemotherapy, and/or radiation. In some specific cases, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

Disclosed herein, in another aspect, is a method of prognosing and/or diagnosing an estrogen receptor positive (ER⁺) breast cancer in an African American subject in need thereof, comprising a. measuring a level of expression of NBN, BRCA1, and one or more of DNA-damage repair (DDR) genes involved in a single strand break repair (SSBR) pathway in a sample from the African American subject; and b. comparing the level of expression to a control level of expression to obtain a comparison expression value, thereby prognosing and/or diagnosing the ER⁺ breast cancer in the African American subject in need thereof. In some cases, the level of expression of NBN and BRCA1 is higher than the control level of expression, and the one or more of DNA-damage-repair-related genes involved in the SSBR pathway is lower or higher than the control level of expression.

In some cases, the method described herein further comprises obtaining the sample from the African American subject in need thereof. In some cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

In some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

In some cases, the method described herein further comprises prognosing the African American subject in need thereof as later having poor disease-specific survival rate, relapse-free survival rate, and/or overall survival rate.

In some cases, the method described herein further comprises administering to the African American subject in need thereof an effective amount of cyclin-dependent kinase inhibitor, endocrine therapy, surgery, chemotherapy, and/or radiation.

In some cases, the African American subject in need thereof has a stage I, II, III, or IV breast cancer. In other cases, the African American subject in need thereof has a node status of 0, 1, 2, 3, or 3+. In other cases, the African American subject in need thereof has a breast cancer that has tested positive or negative for progesterone receptors. In other cases, the African American subject in need thereof has a breast cancer that has tested positive or negative for HER2 protein.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative aspects.

FIGS. 1A-1D depict a schematic of downregulation of DDR genes that associate with poor outcome in CA ER+ breast cancer patients do not associate with poor outcome in AAs. (FIG. 1A-1C) Bubbleplots representing % tumors from AA or CA patients with low RNA levels (mean-1.5*std devn) of DDR genes belonging to nucleotide excision repair (NER: CETN2, ERCC1), mismatch repair (MMR: MLH1, PMS2), or both (Multiple) in dataset #1 (A), dataset #2 (B) and TCGA (C). (FIG. 1D) Column graph depicting cumulative % of AA and CA patients with disease recurrence, from all three datasets, whose tumors either had detectable dysregulation of any DDR genes or did not. Pearson's Chi Squared test determined all p-values. Associated descriptive characteristics of each of the three datasets can be seen in Tables 2-4.

FIGS. 2A-2H show that AA tumors have a distinct pattern of DDR dysregulation. (FIG. 2A) Venn diagram showing proportion of genes from each of six DDR pathways (listed in Table 1) that are significantly (q<0.25) dysregulated in AA tumors relative to CAs in at least one of three datasets analyzed. Schematic of analysis in FIG. 6 Associated FIG. 7 presents full list of DDR genes. (FIG. 2B) Stacked column graphs representing number of DDR genes that are up (yellow) or down (blue)-regulated by pathway. Pearson's chi-squared test determined p-values. (FIG. 2C) Heatmap showing candidate genes that are either up-(yellow) or down-regulated (blue) in at least two datasets as assessed by q-value analysis based on p-values derived from Wilcoxon Rank Sum tests. Associated FIGS. 8-9 present boxplots of each candidate gene described in the heatmap. (FIG. 2D) Bar graph showing observed/expected ratio for mutational frequency of all DDR genes mutated at least once in AA patient tumors from TCGA. Observed/expected ratio for CA tumors are presented by bars to the left of the median line, while the ratio for AAs is presented to the right. Pearson's chi-squared test determined p-values. Associated FIG. 10 presents additional data on DDR gene mutations in AA and CA tumors. (FIG. 2E) Layered donut plot depicting proportional representation of functional DNA repair categories in the 104 DDR genes screened (outer donut) and in the list of 12 genes identified in (C) and (D) (inner donut). Fisher's exact test determined p-value. ATM/Chk2 and ATR/Chk1 scaffolds were combined to represent cell cycle checkpoint scaffolds, as a distinct category from repair-scaffolds. Full list of genes in functional categories presented in Table 1. NER, Nucleotide excision repair, BER, Base excision repair, MMR, mismatch repair, NHEJ, non-homologous end joining, FA, Fanconi anemia, HR, homologous recombination, SSBR, single strand break repair, DSBR, double strand break repair. #1, dataset #1, #2, dataset #2. (FIG. 2F) Representation of functional DNA repair categories in 104 DDR genes screened and in the list of 8 genes. Fisher's exact test determined p-values. ATM/Chk2 and ATR/Chk1 scaffolds were combined to represent cell cycle checkpoint scaffolds in statistical analyses. (FIG. 2G) Heat map demonstrating up-(yellow) or down-(blue)-regulation of the four principle CDKs in tumors from #958 and TCGA with dysregulation of the 8 shortlisted DDR genes, grouped based on pathway as indicated. The third dataset was not included in this analysis as it is missing RNA data from all CDK genes. Non-HR and non-BER genes were grouped as Other DDR. Asterisks indicate statistical significance (p<0.05*, p<0.01**, p<0.001***) from two-tailed Wilcoxon Rank Sum tests after Holm's adjustment for multiple comparisons) and outlined boxes indicate genes with consistent statistical significance across datasets. Schematic of the cell cycle and checkpoint kinases included for context. (FIG. 211) PCNA RNA analysis in TCGA. Multivariate ANOVA test with pairwise comparison determined p-value. NER, Nucleotide excision repair, BER, Base excision repair, MMR, mismatch repair, NHEJ, non-homologous end joining, FA, Fanconi anemia, HR, homologous recombination, SSBR, single strand break repair, DSBR, double strand break repair, ns, not significant.

FIGS. 3A-3F show differences in the DDR landscape of AA vs CA normal breast tissue. (FIG. 3A). Nested donut plots representing proportion of SSBR (blue) vs DSBR (yellow) genes (outer donut) and proportion of genes from each pathway within SSBR and DSBR categories (inner donut) that were significantly (p<0.1) up- or down-regulated in AA vs CA normal breast samples. Wilcoxon Rank Sum test determined p-values. Distribution of DDR dysregulation in tumor datasets analyzed in FIG. 2 is presented for comparison. (FIG. 3B) Stacked column graph representing #genes by pathway that are significantly (p<0.1) up (yellow) or down (blue) regulated in AA relative to CA normal breast tissue. (FIG. 3C) Stacked column graphs summarizing numbers of DSBR that are either up-(yellow) or down-(blue) regulated in normal (Normal and Adj) vs tumor (#1, #2, TCGA) datasets. (FIG. 3D) Stacked column graphs summarizing numbers of SSBR genes that are either up-(yellow) or down-(blue) regulated in normal (Normal and Adj) vs tumor (#1, #2, TCGA) datasets (FIG. 3E) Stacked column graphs representing proportion of high and low proliferating samples. High proliferating samples reflect the top 20% of tumors ranked by gene expression of MKI67, a marker of proliferation from high to low, while low proliferating samples reflect the bottom 20th percentile. (FIG. 3F) Stacked column graphs representing proportion of and pre- and post-menopausal AA and CA women in normal vs tumor datasets. Pearson's chi-squared test determined all categorical p-values. NER, Nucleotide excision repair, BER, Base excision repair, MMR, mismatch repair, NHEJ, non-homologous end joining, FA, Fanconi anemia, HR, homologous recombination, SSBR, single strand break repair, DSBR, double strand break repair. #1, dataset 1, #2, dataset 2, Adj,tumor-adjacent normal, Norm, normal.

FIGS. 4A-4F show DDR genes enriched for dysregulation in AA tumors associate with worse survival. (FIGS. 4A-4C) Stacked columns representing % of AA vs CA tumors with dysregulation of any one of the DDR genes from the specified pathways in dataset #1 (A), dataset #2 (B) and TCGA (C). Pearson's chi-squared test determined p-values. Associated data presented in FIGS. 11A-C. (FIG. 4D-4F) Kaplan-Meier curves representing disease-specific survival in dataset #1 (D), relapse-free survival in dataset #2 (E) and overall survival in TCGA (F) associated with patients whose tumors had dysregulation of specified DDR genes by pathway relative to tumors that did not. Log rank test determined p-values. Associated data in FIGS. 11D-F, and Cox Regression Proportional Hazards assessment in Tables 5-7. HR in survival curves, hazard ratio, p<0.05, *; p<0.01, **; p<0.001, ***.

FIGS. 5A-5B show major breast cancer patient tumor datasets underrepresent AAs. (FIG. 5A) Stacked column graphs representing the number of AA (red) and CA (black) patient tumors in a breast cancer dataset TCGA. (FIG. 5B) Stacked column graphs representing the number of AA (red) and CA (black) patient tumors in a breast cancer dataset METABRIC. Numbers of AA and CA patients diagnosed with breast cancer per 1000 diagnoses in the USA in 2019 are included for comparison.

FIGS. 6A-6C show schematic of study and a working model. (FIG. 6A) Outline and details of datasets used in the study and order in which analyses were conducted and are described. (FIG. 6B) Three tumor and two normal breast datasets were analyzed for differences in RNA levels of 104 DNA damage repair (DDR) genes from 6 pathways to map the landscape of DDR in Black (B) vs white (W) women. DDR genes differently expressed in tumors from Black women in >1 tumor dataset were assessed in survival analyses in all three datasets. In parallel, differences in RNA levels of DDR genes in Black vs white women were interrogated at pathway level in two normal breast datasets. (FIG. 6C) Working model proposing co-incidence of DSBR upregulation (primarily, of HR genes) in normal breast and up- or down-regulation of SSBR genes (primarily down-regulation of BER genes) during tumor formation and progression, in ER+ tumors from Black women. This co-incident altered DDR regulation associates with increased CDK2 gene expression, and significantly worse survival outcome. NER, Nucleotide excision repair, BER, Base excision repair, MMR, mismatch repair, NHEJ, non-homologous end joining, FA, Fanconi anemia, HR, homologous recombination, SSBR, single strand break repair, DSBR, double strand break repair, N/A, statistical analysis not available because of small sample size or insufficient follow up, ns, not significant.

FIGS. 7A-7B show landscape of DDR dysregulation specific to AA tumors. (FIG. 7A) Heatmap showing all single stranded break repair (SSBR) genes that had significantly different RNA levels in tumors from AA patients when compared to tumors from CAs in at least one dataset. (FIG. 7B) Heatmap showing all double strand break repair (DSBR) genes that had significantly different RNA levels in tumors from AA patients when compared to tumors from CAs in at least one dataset. Genes were considered to have significantly different RNA levels if they had a q<0.25 based on p-values generated by comparing RNA levels of 104 DDR genes unique to 6 common DDR pathways using Wilcoxon Rank Sum test. RNA levels are denoted as higher in AA tumors (yellow), lower (blue) or missing (grey). Supports data presented in FIG. 2.

FIG. 8 shows dysregulation of candidate DDR genes from BER and NER pathways. Boxplots showing RNA levels of each DDR gene that was dysregulated in at least two of the three tumor datasets analyzed. Wilcoxon Rank Sum test determined p-values. Horizontal line depicts the median and error bars the standard deviation.

FIG. 9 shows dysregulation of candidate DDR genes from NHEJ, FA, and HR pathways. Boxplots showing RNA levels of each DDR gene that was dysregulated in at least two of the three tumor datasets analyzed. Wilcoxon Rank Sum test determined p-values. Horizontal line depicts the median and error bars the standard deviation.

FIGS. 10A-10C show mutational landscape of DDR genes in AA tumors. (FIG. 10A) Oncoprint from cBio depicting mutations in candidate genes identified in the analysis in FIG. 2 in AA (red) and CA (blue) ER+ patient tumors from TCGA. Boxes indicate statistical significance (p<0.05). Green indicates missense and black, nonsense mutations; (FIG. 10B) Stacked column graph representing the proportion of ER+ tumors in AA vs CA patients with mutations in any DDR gene in the specified pathways. Pearson's Chi Squared test determined p-values. (FIG. 10C) Kaplan-Meier disease free survival curves for AA and CA ER⁺ patients whose tumors harbored mutations in any DDR gene. Analysis was restricted to genes that were mutated at least once in AA tumors. Log rank test determined p-values. HR, hazard ratios for survival curves, and homologous recombination in context of DDR pathways.

FIGS. 11A-11F show association of AA-specific DDR dysregulation with patient outcomes. (FIGS. 11A-11C) Stacked columns representing the percentage of AA vs CA tumors with dysregulation of any DDR genes from the specified pathways in dataset #1 (A), dataset #2 (B) and TCGA (C). Pearson's chi-squared test determined p-values for overall distribution of DDR gene dysregulation in AAs vs CAs, and also by individual pathways. Associated data presented in FIGS. 4A-C. (FIGS. 11D-11F) Kaplan-Meier survival curves representing disease-specific survival in dataset #1 (D), relapse-free survival in dataset #2 (E) and overall survival in TCGA (F) associated with patients whose tumors had dysregulation of specified candidate DDR genes by pathway relative to tumors that did not. Log rank test determined p-values. Associated data in FIGS. 4D-F, and Cox Regression Proportional Hazards assessment in Tables 5-7. HR in survival curves, hazard ratio, p<0.05, *; p<0.01, **; p<0.001, ***.

### DETAILED DESCRIPTION

Previous studies have shown a link between DNA damage repair (DDR) dysregulation and endocrine therapy resistance in ER+ breast cancer. However, such studies relied on datasets where African American women were unrepresented. The present invention is based, at least in part, on finding higher frequency dysregulation of DDR genes in African American women, thus leading to resistance to endocrine therapy. High RNA levels of at least five genes, NBN, BRCA1, FANCE, MNAT1 and NEIL3, and low RNA levels of at least three genes XRCC4, XRCC1 and PARP1 occur at 3-5 fold higher frequency in tumors from African American patients relative to Caucasian Americans. Moreover, 10% of African American ER+ tumors have simultaneous dysregulation of multiple of these DNA repair genes, which is observable in only ˜1% of tumors from Caucasian Americans. In addition, the finding of a statistically significant association between the HR/SSBR signature observed in ER+ tumors from Black women and upregulation of CDK2, the cyclin dependent kinase instrumental in S phase progression, indicates that at least a subset of Black ER+ breast cancer patients may benefit from earlier intervention with CDK2 inhibitors in combination with endocrine therapy.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present aspects disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, “of” may refer to “and”, “or,” or “and/of” and may be used both exclusively and inclusively. For example, the term “A or B” may refer to “A or B”, “A but not B”, “B but not A”, and “A and B”. In some cases, context may dictate a particular meaning.

As used herein, the phrases “at least one”, “one or more”, and “and/of” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “increased”, “increasing”, or “increase” are used herein to generally mean an increase by a statically significant amount. In some cases, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. Other examples of “decrease” include a decrease of at least 2-fold, at least 5-fold, at least 10-fold, at least 20-fold, at least 50-fold, at least 100-fold, at least 1000-fold or more as compared to a reference level. The decrease can be, for example, at least 10%, at least 20%, at least 30%, at least 40% or more, and is preferably down to a level accepted as within the range of normal for an individual without a given disease.

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication of a disorder being treated or amelioration of symptoms of a disorder being treated. Also, a therapeutic benefit may be achieved with the eradication or amelioration of one or more physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease can undergo treatment, even though a diagnosis of a disease has not been made.

The terms “effective amount” and “therapeutically effective amount,” are used interchangeably herein and generally refer to a quantity of a pharmaceutical composition, for example a pharmaceutical composition comprising the composition described herein, that is sufficient to result in a desired activity upon administration to a subject in need thereof. Within the context of the present disclosure, the term “therapeutically effective” refers to that quantity of a pharmaceutical composition that is sufficient to delay the manifestation, arrest the progression, relieve or alleviate at least one symptom of a disorder treated by the methods of the present disclosure.

The terms “patient” or “subject” are used interchangeably herein and encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

Methods of Prognosing and/or Diagnosing an ER⁺ Breast Cancer in an African American Subject Using DDR Genes

Described herein are methods of prognosing and/or diagnosing an estrogen receptor positive (ER+) breast cancer in an African American subject in need thereof, comprising: a. measuring a level of expression of one or more DNA damage repair (DDR) genes in a sample from the African American subject, wherein the one or more DDR genes is selected from the group consisting of XPC, XPA, RBX1, RAD23B, RAD23A, MNAT1, GTF2H5, GTF2H4, GTF2H3, GTF2H2, GTF2H1, ERCC8, ERCC6, ERCC5, ERCC4, ERCC3, ERCC2, ERCC1, DDB2, DDB1, CUL4B, CUL4A, CETN2, XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, APEX1, PMS2P3, PMS2, PMS1, MSH6, MSH5, MSH4, MSH3, MSH2, MLH3, MLH1, EXO1, XRCC6, XRCC5, XRCC4, PRKDC, POLM, NHEJ1, LIG4, DNTT, XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, BLM, SLX4, PALB2, FANCM, FANCL, FANCI, FANCG, FANCF, FANCE, FANCD2, FANC, FANCB, FANCA, FAAP24, FAAP20, and BRIP1; and b. comparing the level of expression to a control level of expression of the one or more DDR genes to obtain a comparison expression value, thereby prognosing and/or diagnosing the ER+ breast cancer in the African American subject in need thereof.

In some cases, the method described herein comprises measuring one DDR gene from the list of XPC, XPA, RBX1, RAD23B, RAD23A, MNAT1, GTF2H5, GTF2H4, GTF2H3, GTF2H2, GTF2H1, ERCC8, ERCC6, ERCC5, ERCC4, ERCC3, ERCC2, ERCC1, DDB2, DDB1, CUL4B, CUL4A, CETN2, XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, APEX1, PMS2P3, PMS2, PMS1, MSH6, MSH5, MSH4, MSH3, MSH2, MLH3, MLH1, EXO1, XRCC6, XRCC5, XRCC4, PRKDC, POLM, NHEJ1, LIG4, DNTT, XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, BLM, SLX4, PALB2, FANCM, FANCL, FANCI, FANCG, FANCF, FANCE, FANCD2, FANC, FANCB, FANCA, FAAP24, FAAP20, and BRIP1. In other cases, the method described herein comprises measuring two, three, four, five, six, seven, eight, nine, or ten DDR genes from the above-listed list. In other cases, the method described herein comprises measuring three DDR genes from the above-listed list. In other cases, the method described herein comprises measuring eleven, twelve, thirteen, fourteen, fifteen, sixteen, seventeen, eighteen, nineteen, or twenty DDR genes from the above-listed list. In other cases, the method described herein comprises measuring twenty-one, twenty-two, twenty-three, twenty-four, twenty-five, twenty-six, twenty-seven, twenty-eight, twenty-nine, or thirty DDR genes from the above-listed list. In other cases, the method described herein comprises measuring thirty-one, thirty-two, thirty-three, thirty-four, thirty-five, thirty-six, thirty-seven, thirty-eight, thirty-nine, or forty DDR genes from the above-listed list. In other cases, the method described herein comprises measuring forty-one, forty-two, forty-three, forty-four, forty-five, forty-six, forty-seven, forty-eight, forty-nine, or fifty DDR genes from the above-listed list. In other cases, the method described herein comprises measuring fifty-one, fifty-two, fifty-three, fifty-four, fifty-five, fifty-six, fifty-seven, fifty-eight, fifty-nine, or sixty DDR genes from the above-listed list. In other cases, the method described herein comprises measuring sixty-one, sixty-two, sixty-three, sixty-four, sixty-five, sixty-six, or all sixty-seven DDR genes from the above-listed list.

In some cases, the method described herein comprises measuring the level of expression of one or more DDR genes encoding cell cycle checkpoint scaffold proteins, which are specified in Table 1. Accordingly, in some cases, the method described herein comprises measuring the level of expression of one or more DDR genes, wherein the one or more DDR genes is selected from the group consisting of XPC, XPA, MNAT1, GTF2H5, GTF2H4, GTF2H3, GTF2H2, GTF2H1, ERCC3, ERCC2, DDB2, DDB1, CUL4B, CUL4A, CETN2, XRCC1, SMUG1, MUTYH, MPG, PMS2P3, PMS2, PMS1, MSH6, MSH3, MSH2, MLH1, EXO1, XRCC5, XRCC4, NHEJ1, LIG4, XRCC3, XRCC2, SHFM1, RAD51D, RAD51B, RAD51, NBN, EME2, BRCA1, SLX4, FANCM, FANCL, FANCI, FANCG, FANCF, FANCE, FANCC, FANCB, FANCA, FAAP20, and BRIP1. In some specific cases, the method described herein comprises measuring two, three, four, five, six, seven, eight, nine, or ten DDR genes from the above-listed list. In other cases, the method described herein comprises measuring three DDR genes from the above-listed list. In other cases, the method described herein comprises measuring eleven, twelve, thirteen, fourteen, fifteen, sixteen, seventeen, eighteen, nineteen, or twenty DDR genes from the above-listed list. In other cases, the method described herein comprises measuring twenty-one, twenty-two, twenty-three, twenty-four, twenty-five, twenty-six, twenty-seven, twenty-eight, twenty-nine, or thirty DDR genes from the above-listed list. In other cases, the method described herein comprises measuring thirty-one, thirty-two, thirty-three, thirty-four, thirty-five, thirty-six, thirty-seven, thirty-eight, thirty-nine, or forty DDR genes from the above-listed list. In other cases, the method described herein comprises measuring forty-one, forty-two, forty-three, forty-four, forty-five, forty-six, forty-seven, forty-eight, forty-nine, or fifty DDR genes from the above-listed list. In other cases, the method described herein comprises measuring fifty-one or fifty-two DDR genes from the above-listed list.

In some cases, the level of expression is up- or down-regulated compared to the control level of expression. In some specific cases, the level of expression is up-regulated by being at least 110%, 120%, 130%, 140%, 150%, 160%, 170%, 180%, 190%, or 200% of the control level of expression. In some specific cases, the level of expression is up-regulated by being at least 210%, 220%, 230%, 240%, 250%, 260%, 270%, 280%, 290%, or 300% of the control level of expression. In some specific cases, the level of expression is up-regulated by being at least 310%, 320%, 330%, 340%, 350%, 360%, 370%, 380%, 390%, or 400% of the control level of expression. In some specific cases, the level of expression is up-regulated by being at least 410%, 420%, 430%, 440%, 450%, 460%, 470%, 480%, 490%, or 500% of the control level of expression. In some specific cases, the level of expression is up-regulated by being at least 550%, 600%, 650%, 700%, 750%, 800%, 850%, 900%, 950%, or 1000% of the control level of expression. In some specific cases, the level of expression is down-regulated by being at most 99%, 98%, 97%, 96%, 95%, 94%, 93%, 92%, 91%, or 90% of the control level of expression. In some specific cases, the level of expression is down-regulated by being at most 89%, 88%, 87%, 86%, 85%, 84%, 83%, 82%, 81%, or 80% of the control level of expression. In some specific cases, the level of expression is down-regulated by being at most 79%, 78%, 77%, 76%, 75%, 74%, 73%, 72%, 71%, or 70% of the control level of expression. In some specific cases, the level of expression is down-regulated by being at most 69%, 68%, 67%, 66%, 65%, 64%, 63%, 62%, 61%, or 60% of the control level of expression. In some specific cases, the level of expression is down-regulated by being at most 59%, 58%, 57%, 56%, 55%, 54%, 53%, 52%, 51%, or 50% of the control level of expression. In some specific cases, the level of expression is down-regulated by being at most 49%, 48%, 47%, 46%, 45%, 44%, 43%, 42%, 41%, or 40% of the control level of expression. In some specific cases, the level of expression is down-regulated by being at most 39%, 38%, 37%, 36%, 35%, 34%, 33%, 32%, 31%, or 30% of the control level of expression. In some specific cases, the level of expression is down-regulated by being at most 29%, 28%, 27%, 26%, 25%, 24%, 23%, 22%, 21%, or 20% of the control level of expression. In some specific cases, the level of expression is down-regulated by being at most 19%, 18%, 17%, 16%, 15%, 14%, 13%, 12%, 11%, or 10% of the control level of expression. In some specific cases, the level of expression is down-regulated by being at most 9%, 8%, 7%, 6%, 5%, 4%, 3%, 2%, or 1% of the control level of expression.

In some cases, the method described herein further comprises obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After obtaining the sample, in some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the level of expression of the one or more of DDR genes listed in the present section is up-regulated by being at least 110%, 120%, 130%, 140%, 150%, 160%, 170%, 180%, 190%, or 200% of the control level of expression, the subject is prognosed as having poor disease-specific survival rate, relapse-free survival rate, and/or overall survival rate. In some specific cases, the prognosing threshold is set so that if the level of expression is up-regulated by being at least 210%, 220%, 230%, 240%, 250%, 260%, 270%, 280%, 290%, or 300% of the control level of expression, the subject is prognosed as having poor disease-specific survival rate, relapse-free survival rate, and/or overall survival rate. In some specific cases, the prognosing threshold is set so that if the level of expression is up-regulated by being at least 310%, 320%, 330%, 340%, 350%, 360%, 370%, 380%, 390%, or 400% of the control level of expression, the subject is prognosed as having poor disease-specific survival rate, relapse-free survival rate, and/or overall survival rate. In some specific cases, the prognosing threshold is set so that if the level of expression is up-regulated by being at least 410%, 420%, 430%, 440%, 450%, 460%, 470%, 480%, 490%, or 500% of the control level of expression, the subject is prognosed as having poor disease-specific survival rate, relapse-free survival rate, and/or overall survival rate. In some specific cases, the prognosing threshold is set so that if the level of expression is up-regulated by being at least 550%, 600%, 650%, 700%, 750%, 800%, 850%, 900%, 950%, or 1000% of the control level of expression, the subject is prognosed as having poor disease-specific survival rate, relapse-free survival rate, and/or overall survival rate. In some specific cases, the prognosing threshold is set so that if the level of expression is down-regulated by being at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression is down-regulated by being at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression is down-regulated by being at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression is down-regulated by being at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression is down-regulated by being at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression is down-regulated by being at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression is down-regulated by being at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression is down-regulated by being at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression is down-regulated by being at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression is down-regulated by being at most 9%, 8%, 7%, 6%, 5%, 4%, 3%, 2%, or 1%, of the control level of expression, the subject is prognosed as having poor disease-specific survival rate, relapse-free survival rate, and/or overall survival rate.

In some cases, the method described herein further comprises administering to the African American subject in need thereof an effective amount of cyclin-dependent kinase inhibitor. In some specific cases, the cyclin-dependent kinase inhibitor is one or more inhibitors of cyclin-dependent kinase 1 (CDK-1), CDK-4, and CDK-6. In other cases, the method described herein further comprises administering to the African American subject in need thereof endocrine therapy, surgery, chemotherapy, and/or radiation.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

Methods of Prognosing and/or Diagnosing an ER⁺ Breast Cancer in an African American Subject Using Specific Genes

Described herein are methods of prognosing and/or diagnosing an estrogen receptor positive (ER⁺) breast cancer in an African American subject in need thereof, comprising measuring a level of expression of one or more of NBN, BRCA1, FANCE, MNAT1, NEIL3, XRCC4, XRCC1, and PARP1 in a sample from the African American subject; and comparing the level of expression to a control level of expression to obtain a comparison expression value, thereby prognosing and/or diagnosing the ER⁺ breast cancer in the African American subject in need thereof.

In some cases, the level of expression of one or more of NBN, BRCA1, FANCE, MNAT1, and NEIL3 is higher than the control level of expression. In specific cases, the level of expression of one or more of NBN, BRCA1, FANCE, MNAT1, and NEIL3 is at least 110%, 120%, 130%, 140%, 150%, 160%, 170%, 180%, 190%, or 200% the control level of expression.

In other specific cases, the level of expression of one or more of NBN, BRCA1, FANCE, MNAT1, and NEIL3 is at least 210%, 220%, 230%, 240%, 250%, 260%, 270%, 280%, 290%, or 300% the control level of expression. In other specific cases, the level of expression of one or more of NBN, BRCA1, FANCE, MNAT1, and NEIL3 is at least 310%, 320%, 330%, 340%, 350%, 360%, 370%, 380%, 390%, or 400% the control level of expression. In other specific cases, the level of expression of one or more of NBN, BRCA1, FANCE, MNAT1, and NEIL3 is at least 410%, 420%, 430%, 440%, 450%, 460%, 470%, 480%, 490%, or 500% the control level of expression. In other specific cases, the level of expression of one or more of NBN, BRCA1, FANCE, MNAT1, and NEIL3 is at least 550%, 600%, 650%, 700%, 750%, 800%, 850%, 900%, 950%, or 1000% the control level of expression.

In some cases, the level of expression of one or more of XRCC4, XRCC1, and PARP1 is lower than the control level of expression. In specific cases, the level of expression of one or more of XRCC4, XRCC1, and PARP1 is at most 99%, 98%, 97%, 96%, 95%, 94%, 93%, 92%, 91%, or 90% of the control level of expression. In specific cases, the level of expression of one or more of XRCC4, XRCC1, and PARP1 is at most 89%, 88%, 87%, 86%, 85%, 84%, 83%, 82%, 81%, or 80% of the control level of expression. In specific cases, the level of expression of one or more of XRCC4, XRCC1, and PARP1 is at most 79%, 78%, 77%, 76%, 75%, 74%, 73%, 72%, 71%, or 70% of the control level of expression. In specific cases, the level of expression of one or more of XRCC4, XRCC1, and PARP1 is at most 69%, 68%, 67%, 66%, 65%, 64%, 63%, 62%, 61%, or 60% of the control level of expression. In specific cases, the level of expression of one or more of XRCC4, XRCC1, and PARP1 is at most 59%, 58%, 57%, 56%, 55%, 54%, 53%, 52%, 51%, or 50% of the control level of expression. In specific cases, the level of expression of one or more of XRCC4, XRCC1, and PARP1 is at most 49%, 48%, 47%, 46%, 45%, 44%, 43%, 42%, 41%, or 40% of the control level of expression. In specific cases, the level of expression of one or more of XRCC4, XRCC1, and PARP1 is at most 39%, 38%, 37%, 36%, 35%, 34%, 33%, 32%, 31%, or 30% of the control level of expression. In specific cases, the level of expression of one or more of XRCC4, XRCC1, and PARP1 is at most 29%, 28%, 27%, 26%, 25%, 24%, 23%, 22%, 21%, or 20% of the control level of expression. In specific cases, the level of expression of one or more of XRCC4, XRCC1, and PARP1 is at most 19%, 18%, 17%, 16%, 15%, 14%, 13%, 12%, 11%, or 10% of the control level of expression. In specific cases, the level of expression of one or more of XRCC4, XRCC1, and PARP1 is at most 9%, 8%, 7%, 6%, 5%, 4%, 3%, 2%, or 1% of the control level of expression.

In some cases, the method described herein comprises measuring a level of expression of one of NBN, BRCA1, FANCE, MNAT1, NEIL3, XRCC4, XRCC1, and PARP1. In other cases, the method described herein comprises measuring a level of expression of two of NBN, BRCA1, FANCE, MNAT1, NEIL3, XRCC4, XRCC1, and PARP1. In other cases, the method described herein comprises measuring a level of expression of three of NBN, BRCA1, FANCE, MNAT1, NEIL3, XRCC4, XRCC1, and PARP1. In other cases, the method described herein comprises measuring a level of expression of four of NBN, BRCA1, FANCE, MNAT1, NEIL3, XRCC4, XRCC1, and PARP1. In other cases, the method described herein comprises measuring a level of expression of five of NBN, BRCA1, FANCE, MNAT1, NEIL3, XRCC4, XRCC1, and PARP1. In other cases, the method described herein comprises measuring a level of expression of six of NBN, BRCA1, FANCE, MNAT1, NEIL3, XRCC4, XRCC1, and PARP1. In other cases, the method described herein comprises measuring a level of expression of seven of NBN, BRCA1, FANCE, MNAT1, NEIL3, XRCC4, XRCC1, and PARP1. In other cases, the method described herein comprises measuring a level of expression of all of NBN, BRCA1, FANCE, MNAT1, NEIL3, XRCC4, XRCC1, and PARP1.

In some cases, the method described herein further comprises obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After obtaining the sample, in some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 99%, 98%, 97%, 96%, 95%, 94%, 93%, 92%, 91%, or 90% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 89%, 88%, 87%, 86%, 85%, 84%, 83%, 82%, 81%, or 80% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 79%, 78%, 77%, 76%, 75%, 74%, 73%, 72%, 71%, or 70% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 69%, 68%, 67%, 66%, 65%, 64%, 63%, 62%, 61%, or 60% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 59%, 58%, 57%, 56%, 55%, 54%, 53%, 52%, 51%, or 50% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 49%, 48%, 47%, 46%, 45%, 44%, 43%, 42%, 41%, or 40% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 39%, 38%, 37%, 36%, 35%, 34%, 33%, 32%, 31%, or 30% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 29%, 28%, 27%, 26%, 25%, 24%, 23%, 22%, 21%, or 20% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 19%, 18%, 17%, 16%, 15%, 14%, 13%, 12%, 11%, or 10% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 110%, 120%, 130%, 140%, 150%, 160%, 170%, 180%, 190%, or 200% of the control level of expression, and/or if the level of expression of one or more of XRCC4, XRCC1, and PARP1 is at most 9%, 8%, 7%, 6%, 5%, 4%, 3%, 2%, or 1% of the control level of expression, subject is prognosed as having poor disease-specific survival rate, relapse-free survival rate, and/or overall survival rate.

In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 99%, 98%, 97%, 96%, 95%, 94%, 93%, 92%, 91%, or 90% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 89%, 88%, 87%, 86%, 85%, 84%, 83%, 82%, 81%, or 80% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 79%, 78%, 77%, 76%, 75%, 74%, 73%, 72%, 71%, or 70% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 69%, 68%, 67%, 66%, 65%, 64%, 63%, 62%, 61%, or 60% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 59%, 58%, 57%, 56%, 55%, 54%, 53%, 52%, 51%, or 50% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 49%, 48%, 47%, 46%, 45%, 44%, 43%, 42%, 41%, or 40% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 39%, 38%, 37%, 36%, 35%, 34%, 33%, 32%, 31%, or 30% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 29%, 28%, 27%, 26%, 25%, 24%, 23%, 22%, 21%, or 20% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 19%, 18%, 17%, 16%, 15%, 14%, 13%, 12%, 11%, or 10% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 210%, 220%, 230%, 240%, 250%, 260%, 270%, 280%, 290%, or 300% of the control level of expression, and/or if the level of expression of one or more of XRCC4, XRCC1, and PARP1 is at most 9%, 8%, 7%, 6%, 5%, 4%, 3%, 2%, or 1% of the control level of expression, subject is prognosed as having poor disease-specific survival rate, relapse-free survival rate, and/or overall survival rate.

In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 99%, 98%, 97%, 96%, 95%, 94%, 93%, 92%, 91%, or 90% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 89%, 88%, 87%, 86%, 85%, 84%, 83%, 82%, 81%, or 80% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 79%, 78%, 77%, 76%, 75%, 74%, 73%, 72%, 71%, or 70% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 69%, 68%, 67%, 66%, 65%, 64%, 63%, 62%, 61%, or 60% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 59%, 58%, 57%, 56%, 55%, 54%, 53%, 52%, 51%, or 50% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 49%, 48%, 47%, 46%, 45%, 44%, 43%, 42%, 41%, or 40% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 39%, 38%, 37%, 36%, 35%, 34%, 33%, 32%, 31%, or 30% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 29%, 28%, 27%, 26%, 25%, 24%, 23%, 22%, 21%, or 20% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 19%, 18%, 17%, 16%, 15%, 14%, 13%, 12%, 11%, or 10% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 310%, 320%, 330%, 340%, 350%, 360%, 370%, 380%, 390%, or 400% of the control level of expression, and/or if the level of expression of one or more of XRCC4, XRCC1, and PARP1 is at most 9%, 8%, 7%, 6%, 5%, 4%, 3%, 2%, or 1% of the control level of expression, subject is prognosed as having poor disease-specific survival rate, relapse-free survival rate, and/or overall survival rate.

In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 99%, 98%, 97%, 96%, 95%, 94%, 93%, 92%, 91%, or 90% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 89%, 88%, 87%, 86%, 85%, 84%, 83%, 82%, 81%, or 80% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 79%, 78%, 77%, 76%, 75%, 74%, 73%, 72%, 71%, or 70% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 69%, 68%, 67%, 66%, 65%, 64%, 63%, 62%, 61%, or 60% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 59%, 58%, 57%, 56%, 55%, 54%, 53%, 52%, 51%, or 50% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 49%, 48%, 47%, 46%, 45%, 44%, 43%, 42%, 41%, or 40% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 39%, 38%, 37%, 36%, 35%, 34%, 33%, 32%, 31%, or 30% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 29%, 28%, 27%, 26%, 25%, 24%, 23%, 22%, 21%, or 20% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 19%, 18%, 17%, 16%, 15%, 14%, 13%, 12%, 11%, or 10% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 410%, 420%, 430%, 440%, 450%, 460%, 470%, 480%, 490%, or 500% of the control level of expression, and/or if the level of expression of one or more of XRCC4, XRCC1, and PARP1 is at most 9%, 8%, 7%, 6%, 5%, 4%, 3%, 2%, or 1% of the control level of expression, subject is prognosed as having poor disease-specific survival rate, relapse-free survival rate, and/or overall survival rate.

In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 99%, 98%, 97%, 96%, 95%, 94%, 93%, 92%, 91%, or 90% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 89%, 88%, 87%, 86%, 85%, 84%, 83%, 82%, 81%, or 80% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 79%, 78%, 77%, 76%, 75%, 74%, 73%, 72%, 71%, or 70% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 69%, 68%, 67%, 66%, 65%, 64%, 63%, 62%, 61%, or 60% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 59%, 58%, 57%, 56%, 55%, 54%, 53%, 52%, 51%, or 50% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 49%, 48%, 47%, 46%, 45%, 44%, 43%, 42%, 41%, or 40% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 39%, 38%, 37%, 36%, 35%, 34%, 33%, 32%, 31%, or 30% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 29%, 28%, 27%, 26%, 25%, 24%, 23%, 22%, 21%, or 20% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 19%, 18%, 17%, 16%, 15%, 14%, 13%, 12%, 11%, or 10% of the control level of expression, subject is prognosed as having poor disease-specific survival rate, relapse-free survival rate, and/or overall survival rate. In some specific cases, the prognosing threshold is set so that if the level of expression of one or more of NBN, BRCA1, FANCE, MNAT1, and NEIL3 is at least 550%, 600%, 650%, 700%, 750%, 800%, 850%, 900%, 950%, or 1000% of the control level of expression, and/or if the level of expression of one or more of XRCC4, XRCC1, and PARP1 is at most 9%, 8%, 7%, 6%, 5%, 4%, 3%, 2%, or 1% of the control level of expression, subject is prognosed as having poor disease-specific survival rate, relapse-free survival rate, and/or overall survival rate.

In some cases, the methods described herein further comprise administering to the African American subject in need thereof an effective amount of cyclin-dependent kinase inhibitor. In other cases, the method described herein further comprises administering to the African American subject in need thereof endocrine therapy, surgery, chemotherapy, and/or radiation.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

Methods of Prognosing and/or Diagnosing an ER+ Breast Cancer in an African American Subject by Identifying Relevant Mutations

Described herein are methods of prognosing and/or diagnosing an estrogen receptor positive (ER⁺) breast cancer in an African American subject in need thereof, comprising identifying mutation(s) in one or more of BRCA1, FAAP24, FANCM, PARP4, PARP1, and ERCC6, thereby prognosing and/or diagnosing the ER⁺ breast cancer in the African American subject in need thereof.

In some cases, the methods described herein comprise identifying mutation(s) in one of BRCA1, FAAP24, FANCM, PARP4, PARP1, and ERCC6. In other cases, the method described herein comprises identifying mutation(s) in two of BRCA1, FAAP24, FANCM, PARP4, PARP1, and ERCC6. In other cases, the method described herein comprises identifying mutation(s) in three of BRCA1, FAAP24, FANCM, PARP4, PARP1, and ERCC6. In other cases, the method described herein comprises identifying mutation(s) in four of BRCA1, FAAP24, FANCM, PARP4, PARP1, and ERCC6. In other cases, the method described herein comprises identifying mutation(s) in five of BRCA1, FAAP24, FANCM, PARP4, PARP1, and ERCC6. In other cases, the method described herein comprises identifying mutation(s) in six of BRCA1, FAAP24, FANCM, PARP4, PARP1, and ERCC6.

In some cases, the methods described herein further comprise obtaining full or partial sequencing information of one or more of BRCA1, FAAP24, FANCM, PARP4, PARP1, and ERCC6. In specific cases, the method described herein further comprises obtaining full or partial sequencing information of one of BRCA1, FAAP24, FANCM, PARP4, PARP1, and ERCC6. In specific cases, the method described herein further comprises obtaining full or partial sequencing information of two of BRCA1, FAAP24, FANCM, PARP4, PARP1, and ERCC6. In specific cases, the method described herein further comprises obtaining full or partial sequencing information of three of BRCA1, FAAP24, FANCM, PARP4, PARP1, and ERCC6. In specific cases, the method described herein further comprises obtaining full or partial sequencing information of four of BRCA1, FAAP24, FANCM, PARP4, PARP1, and ERCC6. In specific cases, the method described herein further comprises obtaining full or partial sequencing information of five of BRCA1, FAAP24, FANCM, PARP4, PARP1, and ERCC6. In specific cases, the method described herein further comprises obtaining full or partial sequencing information of six of BRCA1, FAAP24, FANCM, PARP4, PARP1, and ERCC6.

In some cases, the partial sequencing information is derived from promoter(s) of one or more of BRCA1, FAAP24, FANCM, PARP4, PARP1, and ERCC6. In other cases, the partial sequencing information is derived from enhancer(s) of one or more of BRCA1, FAAP24, FANCM, PARP4, PARP1, and ERCC6. In other cases, the partial sequencing information is derived from coding sequence(s) of one or more of BRCA1, FAAP24, FANCM, PARP4, PARP1, and ERCC6. In some cases, the partial sequencing information is derived from 5′UTR(s) of one or more of BRCA1, FAAP24, FANCM, PARP4, PARP1, and ERCC6. In other cases, the partial sequencing information is derived from 3′ UTR(s) of one or more of BRCA1, FAAP24, FANCM, PARP4, PARP1, and ERCC6.

In some cases, the mutation(s) described herein can be insertion. In other cases, the mutation(s) described herein can be deletion. In other cases, the mutation(s) described herein can be substitution. In other cases, the mutation(s) described herein can be translocation. In other cases, the mutation(s) described herein can be frameshift mutation(s). In other cases, the mutation(s) described herein can be nonsense mutation(s). In other cases, the mutation(s) described herein can be missense mutation(s).

In some cases, the methods described herein further comprise obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the number of mutations in each of the one or more of BRCA1, FAAP24, FANCM, PARP4, PARP1, and ERCC6 is at least one, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least two, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least three, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least four, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least five, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least six, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least seven, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least eight, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least nine, the subject is prognosed as having poor disease-specific survival rate, relapse-free survival rate, and/or overall survival rate. In some specific cases, the prognosing threshold is set so that if the number of mutations in each of the one or more of BRCA1, FAAP24, FANCM, PARP4, PARP1, and ERCC6 is at least ten, the subject is prognosed as having poor disease-specific survival rate, relapse-free survival rate, and/or overall survival rate.

In some cases, the methods described herein further comprise administering to the African American subject in need thereof an effective amount of cyclin-dependent kinase inhibitor. In other cases, the method described herein further comprises administering to the African American subject in need thereof endocrine therapy, surgery, chemotherapy, and/or radiation.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

Methods of Treating an ER⁺ Breast Cancer with CDK2 Inhibitors

Described herein are methods of treating an estrogen receptor positive (ER⁺) breast cancer in a susceptible African American subject, comprising a. identifying the susceptible African American subject by i. measuring a level of expression of one or more of DNA damage repair (DDR) genes involved in a homologous recombination (HR) pathway, wherein the one or more DDR genes involved in the HR pathway are selected from the group consisting of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, and BLM; and a level of expression of one or more of DDR genes involved in a base excision repair (BER) pathway, wherein the one or more DDR genes involved in the BER pathway are selected from the group consisting of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1; and ii. comparing the level of expression to a control level of expression to obtain a comparison expression value; and b. administering to the susceptible African American subject an effective amount of one or more cyclin-dependent kinase 2 (CDK2) inhibitors, thereby treating the ER⁺ breast cancer in the African American subject.

In some cases, the level of expression of one or more of DDR genes involved in the HR pathway is higher than the control level of expression. In specific cases, the level of expression of one or more of DDR genes involved in the HR pathway is at least 110%, 120%, 130%, 140%, 150%, 160%, 170%, 180%, 190%, or 200% of the control level of expression. In specific cases, the level of expression of one or more of DDR genes involved in the HR pathway is at least 210%, 220%, 230%, 240%, 250%, 260%, 270%, 280%, 290%, or 300% of the control level of expression. In specific cases, the level of expression of one or more of DDR genes involved in the HR pathway is at least 310%, 320%, 330%, 340%, 350%, 360%, 370%, 380%, 390%, or 400% of the control level of expression. In specific cases, the level of expression of one or more of DDR genes involved in the HR pathway is at least 410%, 420%, 430%, 440%, 450%, 460%, 470%, 480%, 490%, or 500% of the control level of expression. In specific cases, the level of expression of one or more of DDR genes involved in the HR pathway is at least 550%, 600%, 650%, 700%, 750%, 800%, 850%, 900%, 950%, or 1000% of the control level of expression.

In some cases, the level of expression of one or more of DDR genes involved in the BER pathway is lower than the control level of expression in the susceptible African American subject. In specific cases, the level of expression of one or more of DDR genes involved in the BER pathway is at most 99%, 98%, 97%, 96%, 95%, 94%, 93%, 92%, 91%, or 90% of the control level of expression. In specific cases, the level of expression of one or more of DDR genes involved in the BER pathway is at most 89%, 88%, 87%, 86%, 85%, 84%, 83%, 82%, 81%, or 80% of the control level of expression. In specific cases, the level of expression of one or more of DDR genes involved in the BER pathway is at most 79%, 78%, 77%, 76%, 75%, 74%, 73%, 72%, 71%, or 70% of the control level of expression. In specific cases, the level of expression of one or more of DDR genes involved in the BER pathway is at most 69%, 68%, 67%, 66%, 65%, 64%, 63%, 62%, 61%, or 60% of the control level of expression. In specific cases, the level of expression of one or more of DDR genes involved in the BER pathway is at most 59%, 58%, 57%, 56%, 55%, 54%, 53%, 52%, 51%, or 50% of the control level of expression. In specific cases, the level of expression of one or more of DDR genes involved in the BER pathway is at most 49%, 48%, 47%, 46%, 45%, 44%, 43%, 42%, 41%, or 40% of the control level of expression. In specific cases, the level of expression of one or more of DDR genes involved in the BER pathway is at most 39%, 38%, 37%, 36%, 35%, 34%, 33%, 32%, 31%, or 30% of the control level of expression. In specific cases, the level of expression of one or more of DDR genes involved in the BER pathway is at most 29%, 28%, 27%, 26%, 25%, 24%, 23%, 22%, 21%, or 20% of the control level of expression. In specific cases, the level of expression of one or more of DDR genes involved in the BER pathway is at most 19%, 18%, 17%, 16%, 15%, 14%, 13%, 12%, 11%, or 10% of the control level of expression. In specific cases, the level of expression of one or more of DDR genes involved in the BER pathway is at most 9%, 8%, 7%, 6%, 5%, 4%, 3%, 2%, or 1% of the control level of expression.

In some cases, the methods described herein further comprise obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After obtaining the sample, in some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 110%, 120%, 130%, 140%, 150%, 160%, 170%, 180%, 190%, or 200% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 210%, 220%, 230%, 240%, 250%, 260%, 270%, 280%, 290%, or 300% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 310%, 320%, 330%, 340%, 350%, 360%, 370%, 380%, 390%, or 400% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 410%, 420%, 430%, 440%, 450%, 460%, 470%, 480%, 490%, or 500% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 550%, 600%, 650%, 700%, 750%, 800%, 850%, 900%, 950%, or 1000% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some cases, the CDK2 inhibitors described herein can be AT7519 from Astex Therapeutics. In some cases, the CDK2 inhibitors described herein can be AG-024322 from Pfizer. In some cases, the CDK2 inhibitors described herein can be Dinaciclib. In some cases, the CDK2 inhibitors described herein can be Cyc065 from Cyclacel Pharmaceuticals. In some cases, the CDK2 inhibitors described herein can be Roniciclib. In some cases, the CDK2 inhibitors described herein can be TG02 from Tragara Pharmaceuticals. In some cases, the CDK2 inhibitors described herein can be Milciclib. Other examples of CDK2 inhibitors can be found in Tadesse et al., J. Med. Chem., 2018, Cozza, Pharmaceuticals (Basel). 2017, Cozza et al., Expert Opin Ther Pat. 2012, and Borgo et al., Signal Transduction and Targeted Therapy, 2021, which are incorporated by reference herein in its entirety.

In some embodiments, the method described herein further comprises administering to the African American subject in need thereof endocrine therapy, surgery, chemotherapy, and/or radiation. In some specific cases, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

Methods of Treating an ER⁺ Breast Cancer with a PCNA Inhibitor

Described herein are methods of treating an estrogen receptor positive (ER⁺) breast cancer in a susceptible African American subject, comprising a. identifying the susceptible African American subject by i. measuring a level of expression of one or more of DNA damage repair (DDR) genes involved in a homologous recombination (HR) pathway, wherein the one or more DDR genes involved in the HR pathway are selected from the group consisting of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1, and BLM; and a level of expression of one or more of DDR genes involved in a base excision repair (BER) pathway, wherein the one or more DDR genes involved in the BER pathway are selected from the group consisting of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1; and ii. comparing the level of expression to a control level of expression to obtain a comparison expression value; and b. administering to the susceptible African American subject an effective amount of one or more proliferating cell nuclear antigen (PCNA) inhibitors, thereby treating the ER⁺ breast cancer in the African American subject.

In some cases, the level of expression of one or more of DDR genes involved in the HR pathway is higher than the control level of expression. In specific cases, the level of expression of one or more of DDR genes involved in the HR pathway is at least 110%, 120%, 130%, 140%, 150%, 160%, 170%, 180%, 190%, or 200% of the control level of expression. In specific cases, the level of expression of one or more of DDR genes involved in the HR pathway is at least 210%, 220%, 230%, 240%, 250%, 260%, 270%, 280%, 290%, or 300% of the control level of expression. In specific cases, the level of expression of one or more of DDR genes involved in the HR pathway is at least 310%, 320%, 330%, 340%, 350%, 360%, 370%, 380%, 390%, or 400% of the control level of expression. In specific cases, the level of expression of one or more of DDR genes involved in the HR pathway is at least 410%, 420%, 430%, 440%, 450%, 460%, 470%, 480%, 490%, or 500% of the control level of expression. In specific cases, the level of expression of one or more of DDR genes involved in the HR pathway is at least 550%, 600%, 650%, 700%, 750%, 800%, 850%, 900%, 950%, or 1000% of the control level of expression.

In some cases, the level of expression of one or more of DDR genes involved in the BER pathway is lower than the control level of expression in the susceptible African American subject. In specific cases, the level of expression of one or more of DDR genes involved in the BER pathway is at most 99%, 98%, 97%, 96%, 95%, 94%, 93%, 92%, 91%, or 90% of the control level of expression. In specific cases, the level of expression of one or more of DDR genes involved in the BER pathway is at most 89%, 88%, 87%, 86%, 85%, 84%, 83%, 82%, 81%, or 80% of the control level of expression. In specific cases, the level of expression of one or more of DDR genes involved in the BER pathway is at most 79%, 78%, 77%, 76%, 75%, 74%, 73%, 72%, 71%, or 70% of the control level of expression. In specific cases, the level of expression of one or more of DDR genes involved in the BER pathway is at most 69%, 68%, 67%, 66%, 65%, 64%, 63%, 62%, 61%, or 60% of the control level of expression. In specific cases, the level of expression of one or more of DDR genes involved in the BER pathway is at most 59%, 58%, 57%, 56%, 55%, 54%, 53%, 52%, 51%, or 50% of the control level of expression. In specific cases, the level of expression of one or more of DDR genes involved in the BER pathway is at most 49%, 48%, 47%, 46%, 45%, 44%, 43%, 42%, 41%, or 40% of the control level of expression. In specific cases, the level of expression of one or more of DDR genes involved in the BER pathway is at most 39%, 38%, 37%, 36%, 35%, 34%, 33%, 32%, 31%, or 30% of the control level of expression. In specific cases, the level of expression of one or more of DDR genes involved in the BER pathway is at most 29%, 28%, 27%, 26%, 25%, 24%, 23%, 22%, 21%, or 20% of the control level of expression. In specific cases, the level of expression of one or more of DDR genes involved in the BER pathway is at most 19%, 18%, 17%, 16%, 15%, 14%, 13%, 12%, 11%, or 10% of the control level of expression. In specific cases, the level of expression of one or more of DDR genes involved in the BER pathway is at most 9%, 8%, 7%, 6%, 5%, 4%, 3%, 2%, or 1% of the control level of expression.

In some cases, the methods described herein further comprise obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After obtaining the sample, in some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 110%, 120%, 130%, 140%, 150%, 160%, 170%, 180%, 190%, or 200% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 110%, 120%, 130%, 140%, 150%, 160%, 170%, 180%, 190%, or 200% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 210%, 220%, 230%, 240%, 250%, 260%, 270%, 280%, 290%, or 300% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 210%, 220%, 230%, 240%, 250%, 260%, 270%, 280%, 290%, or 300% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 310%, 320%, 330%, 340%, 350%, 360%, 370%, 380%, 390%, or 400% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 310%, 320%, 330%, 340%, 350%, 360%, 370%, 380%, 390%, or 400% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 410%, 420%, 430%, 440%, 450%, 460%, 470%, 480%, 490%, or 500% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 410%, 420%, 430%, 440%, 450%, 460%, 470%, 480%, 490%, or 500% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of DDR genes involved in the HR pathway from the list of XRCC3, XRCC2, TOP3B, TOP3A, SHFM1, RBBP8, RAD54LRAD54B, RAD52, RAD51D, RAD51B, RAD51, NBN, MUS81, SLX1B, SLX1A, GEN1, EME2, EME1, DMC1, BRACA1 (list 1) is at least 550%, 600%, 650%, 700%, 750%, 800%, 850%, 900%, 950%, or 1000% of the control level of expression, and/or if the level of expression of one or more of DDR genes involved in the BER pathway from the list of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1 (list 2) at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of DDR genes involved in the HR pathway from list 1 is at least 550%, 600%, 650%, 700%, 750%, 800%, 850%, 900%, 950%, or 1000% of the control level of expression, and/or if the level of expression of one or more of DDR genes involved in the BER pathway from list 2 at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some cases, the PCNA inhibitors described herein are selected from those described in Bartolowits et al., ACS Omega 2019, Punchihewa et al., J Biol Chem. 2012, Muller et al., PloS One, 2013, which are incorporated by reference herein in its entirety.

In some embodiments, the methods described herein further comprise administering to the African American subject in need thereof endocrine therapy, surgery, chemotherapy, and/or radiation. In some specific cases, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

Methods of Treating an ER⁺ Breast Cancer with Certain CDK Inhibitors

Described herein are methods of treating an estrogen receptor positive (ER⁺) breast cancer in an African American subject in need thereof comprising administering an effective amount of one or more inhibitors of cyclin-dependent kinase 1 (CDK-1), CDK-4, and CDK-6.

In some cases, the one or more inhibitors of CDK-1, CDK-4, and CDK-6 comprise abemaciclib, Palbociclib, ribociclib, or a combination thereof. In other cases, the one or more inhibitors of CDK-1, CDK-4, and CDK-6 can be found in Zhang et al., Am J Cancer Res. 2021, Asghar et al., Nat Rev Drug Discov. 2015, and Lam et al., US Pharm. 2020, which are incorporated by reference herein in its entirety.

In some cases, the methods described herein further comprise administering to the susceptible African American subject endocrine therapy, surgery, chemotherapy, and/or radiation. In some specific embodiments, the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

Methods of Prognosing and/or Diagnosing an ER⁺ Breast Cancer in an African American Subject by Measuring Genes from HR and SSBR Pathways

Described herein are methods of prognosing and/or diagnosing an estrogen receptor positive (ER+) breast cancer in an African American subject in need thereof, comprising a. measuring a level of expression of NBN, BRCA1, and one or more of DNA-damage repair (DDR) genes involved in a single strand break repair (SSBR) pathway in a sample from the African American subject; and b. comparing the level of expression to a control level of expression to obtain a comparison expression value, thereby prognosing and/or diagnosing the ER⁺ breast cancer in the African American subject in need thereof.

In some cases, the level of expression of NBN and BRCA1 is higher than the control level of expression. In specific cases, the level of expression of NBN and BRCA1 is at least 110%, 120%, 130%, 140%, 150%, 160%, 170%, 180%, 190%, or 200% of the control level of expression. In specific cases, the level of expression of NBN and BRCA1 is at least 210%, 220%, 230%, 240%, 250%, 260%, 270%, 280%, 290%, or 300% of the control level of expression. In specific cases, the level of expression of NBN and BRCA1 is at least 310%, 320%, 330%, 340%, 350%, 360%, 370%, 380%, 390%, or 400% of the control level of expression. In specific cases, the level of expression of NBN and BRCA1 is at least 410%, 420%, 430%, 440%, 450%, 460%, 470%, 480%, 490%, or 500% of the control level of expression. In specific cases, the level of expression of NBN and BRCA1 is at least 550%, 600%, 650%, 700%, 750%, 800%, 850%, 900%, 950%, or 1000% of the control level of expression.

In some cases, the one or more of DDR genes involved in the SSBR pathway is selected from the group consisting of XRCC1, PARP1, NAIL3, and MNAT1. In specific cases, one or both of XRCC1 and PARP1 is lower the control level of expression. In specific cases, the level of expression of XRCC1 and PARP1 is at most 99%, 98%, 97%, 96%, 95%, 94%, 93%, 92%, 91%, or 90% of the control level of expression. In specific cases, the level of expression of XRCC1 and PARP1 is at most 89%, 88%, 87%, 86%, 85%, 84%, 83%, 82%, 81%, or 80% of the control level of expression. In specific cases, the level of expression of XRCC1 and PARP1 is at most 79%, 78%, 77%, 76%, 75%, 74%, 73%, 72%, 71%, or 70% of the control level of expression. In specific cases, the level of expression of XRCC1 and PARP1 is at most 69%, 68%, 67%, 66%, 65%, 64%, 63%, 62%, 61%, or 60% of the control level of expression. In specific cases, the level of expression of XRCC1 and PARP1 is at most 59%, 58%, 57%, 56%, 55%, 54%, 53%, 52%, 51%, or 50% of the control level of expression. In specific cases, the level of expression of XRCC1 and PARP1 is at most 49%, 48%, 47%, 46%, 45%, 44%, 43%, 42%, 41%, or 40% of the control level of expression. In specific cases, the level of expression of XRCC1 and PARP1 is at most 39%, 38%, 37%, 36%, 35%, 34%, 33%, 32%, 31%, or 30% of the control level of expression. In specific cases, the level of expression of XRCC1 and PARP1 is at most 29%, 28%, 27%, 26%, 25%, 24%, 23%, 22%, 21%, or 20% of the control level of expression. In specific cases, the level of expression of XRCC1 and PARP1 is at most 19%, 18%, 17%, 16%, 15%, 14%, 13%, 12%, 11%, or 10% of the control level of expression. In specific cases, the level of expression of XRCC1 and PARP1 is at most 9%, 8%, 7%, 6%, 5%, 4%, 3%, 2%, or 1% of the control level of expression. In specific cases, one or both of NAIL3 and MNAT1 is higher than the control level of expression. In specific cases, the level of expression of NAIL3 and MNAT1 is at least 110%, 120%, 130%, 140%, 150%, 160%, 170%, 180%, 190%, or 200% of the control level of expression. In specific cases, the level of expression of NAIL3 and MNAT1 is at least 210%, 220%, 230%, 240%, 250%, 260%, 270%, 280%, 290%, or 300% of the control level of expression. In specific cases, the level of expression of NAIL3 and MNAT1 is at least 310%, 320%, 330%, 340%, 350%, 360%, 370%, 380%, 390%, or 400% of the control level of expression. In specific cases, the level of expression of NAIL3 and MNAT1 is at least 410%, 420%, 430%, 440%, 450%, 460%, 470%, 480%, 490%, or 500% of the control level of expression. In specific cases, the level of expression of NAIL3 and MNAT1 is at least 550%, 600%, 650%, 700%, 750%, 800%, 850%, 900%, 950%, or 1000% of the control level of expression.

In some cases, the methods described herein further comprise obtaining the sample from the African American subject in need thereof, wherein the type of the sample can be a kind below, and wherein the timing of obtaining the sample can be one of the situations below. In some specific cases, the sample comprises biopsy, urine, feces, polyp, cerebrospinal fluid, blood, semen, nipple aspirate, or a combination thereof. In some cases, the sample is obtained when the African American subject in need thereof is suspected of having breast cancer after breast test, mammogram, and/or breast ultrasound. In other cases, the sample is obtained when the African American subject in need thereof experiences a recurrent or metastatic breast cancer after one or more treatments.

After obtaining the sample, in some cases, the level of expression is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

After measuring the level of expression of relevant genes and comparing to a control level of expression, in some cases, the method described herein further comprises prognosing the African American subject in need thereof as later having poor disease-specific survival rate. In other cases, the method described herein further comprises prognosing the African American subject in need thereof as later having poor relapse-free survival rate. In other cases, the method described herein further comprises prognosing the African American subject in need thereof as later having poor overall survival rate.

In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 110%, 120%, 130%, 140%, 150%, 160%, 170%, 180%, 190%, or 200% of the control level of expression, and/or if the level of expression of the one or more of XRCC1 and PARP1 is at most 9%, 8%, 7%, 6%, 5%, 4%, 3%, 2%, or 1% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 210%, 220%, 230%, 240%, 250%, 260%, 270%, 280%, 290%, or 300% of the control level of expression, and/or if the level of expression of the one or more of XRCC1 and PARP1 is at most 9%, 8%, 7%, 6%, 5%, 4%, 3%, 2%, or 1% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 310%, 320%, 330%, 340%, 350%, 360%, 370%, 380%, 390%, or 400% of the control level of expression, and/or if the level of expression of the one or more of XRCC1 and PARP1 is at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 410%, 420%, 430%, 440%, 450%, 460%, 470%, 480%, 490%, or 500% of the control level of expression, and/or if the level of expression of the one or more of XRCC1 and PARP1 is at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 99%, 98%, 97%, 96%, 95%, 94%, 93%, 92%, 91%, or 90% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 89%, 88%, 87%, 86%, 85%, 84%, 83%, 82%, 81%, or 80% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 79%, 78%, 77%, 76%, 75%, 74%, 73%, 72%, 71%, or 70% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 69%, 68%, 67%, 66%, 65%, 64%, 63%, 62%, 61%, or 60% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 59%, 58%, 57%, 56%, 55%, 54%, 53%, 52%, 51%, or 50% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 49%, 48%, 47%, 46%, 45%, 44%, 43%, 42%, 41%, or 40% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 39%, 38%, 37%, 36%, 35%, 34%, 33%, 32%, 31%, or 30% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 29%, 28%, 27%, 26%, 25%, 24%, 23%, 22%, 21%, or 20% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 19%, 18%, 17%, 16%, 15%, 14%, 13%, 12%, 11%, or 10% of the control level of expression, the subject is prognosed as having poor disease-specific survival rate, relapse-free survival rate, and/or overall survival rate. In some specific cases, the prognosing threshold is set so that if the level of expression of the one or more of NBN, BRCA1, NAIL3, and MNAT1 is at least 550%, 600%, 650%, 700%, 750%, 800%, 850%, 900%, 950%, or 1000% of the control level of expression, and/or if the level of expression of the one or more of XRCC1 and PARP1 is at most 9%, 8%, 7%, 6%, 5%, 4%, 3%, 2%, or 1% of the control level of expression, the subject is prognosed as having poor disease-specific survival rate, relapse-free survival rate, and/or overall survival rate.

In some cases, the methods described herein further comprise administering to the African American subject in need thereof an effective amount of cyclin-dependent kinase inhibitor. In other cases, the method described herein further comprises administering to the African American subject in need thereof endocrine therapy, surgery, chemotherapy, and/or radiation.

In some cases, the African American subject in need thereof has a stage I breast cancer. In other cases, the African American subject in need thereof has a stage II breast cancer. the African American subject in need thereof has a stage III breast cancer. In other cases, the African American subject in need thereof has a stage IV breast cancer.

In some cases, the African American subject in need thereof has a node status of 0. In other cases, the African American subject in need thereof has a node status of 1. In other cases, the African American subject in need thereof has a node status of 2. In other cases, the African American subject in need thereof has a node status of 3. In other cases, the African American subject in need thereof has a node status of larger than 3.

In some cases, the African American subject in need thereof has a breast cancer that has tested positive for progesterone receptors. In other cases, the African American subject in need thereof has a breast cancer that has tested negative for progesterone receptors.

In some cases, the African American subject in need thereof has a breast cancer that has tested positive for HER2 protein. In other cases, the African American subject in need thereof has a breast cancer that has tested negative for HER2 protein.

### EXAMPLES

The following illustrative examples are representative of aspects of the stimulation, systems, and methods described herein and are not meant to be limiting in any way.

### Example 1. Prognosing or Diagnosing Breast Cancer in African American Population

### Material and Methods

DDR gene set compilation: Gene sets for all DDR pathways analyzed (Mismatch repair, Nucleotide excision repair, Base excision repair, Non-homologous end joining, Fanconi Anemia and Homologous recombination) were derived from our previously published, curated DDR gene list (detailed in Table 1). Genes shared across different DDR pathways were not included.

TABLE 1
List of genes with Functional Categories

| Gene Name | DNA repair Pathway | Function |
|---|---|---|
| XPC | Nucleotide excision repair | Lesion recognition, |
| | | recruits ATM/ATR |
| XPA | | Acts as scaffold for ATR |
| | | to bind |
| RBX1 | | Lesion recognition |
| RAD23B | | Lesion recognition |
| RAD23A | | Lesion recognition |
| MNAT1 | | Acts as scaffold |
| | | associates with ATM |
| GTF2H5 | | Acts as scaffold |
| | | associates with ATM |
| GTF2H4 | | Acts as scaffold |
| | | associates with ATM |
| GTF2H3 | | Acts as scaffold |
| | | associates with ATM |
| GTF2H2 | | Acts as scaffold |
| | | associates with ATM |
| GTF2H1 | | Acts as scaffold |
| | | associates with ATM |
| ERCC8 | | Lesion recognition |
| ERCC6 | | Lesion recognition |
| ERCC5 | | Endonuclease/Repair |
| | | activity |
| ERCC4 | | Endonuclease/Repair |
| | | activity |
| ERCC3 | | Acts as Scaffold |
| ERCC2 | | Acts as scaffold |
| ERCC1 | | Endonuclease/Repair |
| | | activity |
| DDB2 | | Lesion recognition/ |
| | | recruits ATM/ATR |
| DDB1 | | Lesion recognition/ |
| | | recruits ATM/ATR |
| CUL4B | | Lesion recognition/ |
| | | recruits ATM/ATR |
| CUL4A | | Lesion recognition/ |
| | | recruits ATM/ATR |
| CETN2 | | Lesion recognition/ |
| | | recruits ATM/ATR |
| XRCC1 | Base Excision Repair | Associates with CHK2 |
| UNG | | Lesion recognition |
| TDG | | Lesion recognition |
| SMUG1 | | Lesion recognition/ |
| | | associates with |
| | | ATM/CHK2 |
| POLB | | Repair activity |
| PNKP | | Repair activity |
| PARP4 | | Repair activity |
| PARP3 | | Repair activity |
| PARP2 | | Repair activity |
| PARP1 | | Lesion recognition/ |
| | | repair activity |
| OGG1 | | Lesion recognition |
| NTHL1 | | Lesion recognition |
| NEIL3 | | Lesion recognition |
| NEIL2 | | Lesion recognition |
| NEIL1 | | Lesion recognition |
| MUTYH | | Lesion recognition/ |
| | | associate with |
| | | ATM/CHK2 |
| MPG | | Lesion recognition/ |
| | | associate with |
| | | ATM/CHK2 |
| MBD4 | | Lesion recognition |
| LIG3 | | Repair activity |
| HMGB1P4 | | Downstream effectors |
| HMGB1P1 | | Downstream effectors |
| HMGB1 | | Downstream effectors |
| APLF | | Downstream effectors |
| APEX2 | | Repair activity |
| APEX1 | | Repair activity |
| PMS2P3 | Mismatch repair pathway | Scaffold |
| PMS2 | | Scaffold/Repair activity |
| PMS1 | | Scaffold |
| MSH6 | | Lesion recognition/ |
| | | Scaffold associates |
| | | with CHK2 |
| MSH5 | | Lesion Recognition |
| MSH4 | | Lesion Recognition |
| MSH3 | | Lesion Recognition/ |
| | | Scaffold associates |
| | | with CHK2 |
| MSH2 | | Lesion Recognition/ |
| | | scaffold associates |
| | | with CHK2 |
| MLH3 | | Lesion Recognition |
| MLH1 | | Repair activity/Scaffold |
| | | associates with ATM |
| EXO1 | | Scaffold |
| XRCC6 | Non-Homologous End Joining | Lesion recognition |
| XRCC5 | | Lesion recognition |
| XRCC4 | | Scaffold |
| PRKDC | | Scaffold |
| POLM | | Downstream effectors |
| NHEI1 | | Scaffold |
| LIG4 | | Scaffold/associates |
| | | with ATM |
| DNTT | | Downstream effectors |
| XRCC3 | Homologous recombination | Scaffold/Repair activity |
| XRCC2 | | Scaffold |
| TOP3B | | Downstream effectors |
| TOP3A | | Downstream effectors |
| SHFM1 | | Scaffold |
| RBBP8 | | Repair activity/ |
| | | endonuclease |
| RAD54L | | Lesion recognition |
| RAD54B | | Lesion recognition |
| RAD52 | | Repair activity |
| RAD51D | | Scaffold |
| RAD51B | | Scaffold |
| RAD51 | | Scaffold |
| NBN | | Lesion recognition/ |
| | | associates with ATM |
| MUSB1 | | Repair activity/ |
| | | endonuclease |
| SLX1B | | Repair activity/ |
| | | endonuclease |
| SLX1A | | Repair activity/ |
| | | endonuclease |
| GEN1 | | Repair activity/ |
| | | endonuclease |
| EME2 | | Scaffold/Repair activity |
| EME1 | | Repair activity/ |
| | | endonuclease |
| DMC1 | | Repair activity/ |
| | | recombinases |
| BRCA1 | | Lesion recognition/ |
| | | Scaffold |
| BLM | | Repair activity |
| SLX4 | Fanconi Anemia | Scaffold |
| PALB2 | | Downstream effectors |
| FANCM | | Scaffold/associates |
| | | with ATR/CHK1 signaling |
| FANCL | | Scaffold/associates |
| | | with ATR/CHK1 signaling |
| FANCI | | Scaffold/associates |
| | | with ATR/CHK1 signaling |
| FANCG | | Scaffold/associates |
| | | with ATR/CHK1 signaling |
| FANCF | | Scaffold/associates |
| | | with ATA/CHK1 signaling |
| FANCE | | Scaffold/associates |
| | | with ATR/CHK1 signaling |
| FANCD2 | | Repair activity |
| FANCC | | Scaffold/associates |
| | | With ATR/CHK1 signaling |
| FANCB | | Scaffold |
| FANCA | | Scaffold/associates |
| | | with ATR/CHK1 signaling |
| FAAP24 | | Lesion recognition |
| FAAP20 | | Scaffold/associates |
| | | with ATR/CHK1 signaling |
| BRIP1 | | Scaffold |

Datasets: Tumor datasets: The first dataset (GE078958, Table 2, referred to henceforth as dataset #1) had microarray data from 51 AA tumors with luminal (predominantly ER+) breast cancer, and 169 CA tumors. The second dataset (GSE18229, Table 3), referred to henceforth as dataset #2) had microarray gene expression data from 44 AA tumors and 85 CA tumors. This dataset included tumors of all subtypes although ˜70% of tumors were ER+/luminal. The third dataset was the subset of ER+ tumors (irrespective of HER2 status) from TCGA and consisted of RNAseq gene expression and whole exome sequence data from 49 AA tumors and 449 CAs (Table 4). These restrictions were used to ensure that each dataset was predominantly ER+/luminal and that there was sufficient sample size of AA tumors. TCGA mutation data (downloaded March 2020) were obtained from cBioPortal. Gene expression from dataset #1 were available through the Gene Expression Omnibus (GEO, GSE78958). Gene expression from dataset #2 were downloaded from dbGaP (downloaded May 2020) and for TCGA (downloaded March 2020) were obtained from cBioPortal. TCGA survival outcomes were downloaded from cBioPortal (downloaded May 2020). Standard cutoffs of mean+/−1.5×SD were used on the RNA data to identify “High” and “Low” subsets respectively in each dataset.

TABLE 2
Patient characteristics from dataset #1

| | | | |
|---|---|---|---|
| Characteristic | CA N = 169(%) | AA N = 51(%) | p value |

| Age | | | | | |
|---|---|---|---|---|---|
| <40 | 14 | (8.3) | 5 | (9.8) | |
| 40-49 | 40 | (23.7) | 13 | (25.5) | |
| >=50 | 114 | (67.5) | 33 | (64.7) | |
| Missing | 1 | (.5) | 0 | | 0.92 |
| Tumor Stage | | | | | |
| I | 75 | (44.4) | 23 | (45.1) | |
| II | 73 | (43.2) | 22 | (43.1) | |
| III | 15 | (8.9) | 5 | (9.8) | |
| IV | 5 | (3) | 1 | (2) | |
| Unknown | 1 | (.5) | 0 | | 0.97 |
| Tumor Grade | | | | | |
| Well | 57 | (33.7) | 9 | (17.6) | |
| Moderate | 81 | (47.9) | 30 | (58.8) | |
| Poor | 30 | (17.8) | 11 | (21.6) | |
| Missing | 1 | (.6) | 1 | (2) | 0.14 |
| ER status | | | | | |
| Positive | 169 | (100) | 50 | (98) | |

| | | | | |
|---|---|---|---|---|
| Negative | 0 | 1 | (2) | 0.07 |

| PR status | | | | | |
|---|---|---|---|---|---|
| Positive | 137 | (81.1) | 39 | (76.5) | |
| Negative | 31 | (18.3) | 12 | (23.5) | |
| Missing | 1 | (.6) | 0 | | 0.62 |
| Subtype | | | | | |
| Lum A | 141 | (83.4) | 42 | (82.4) | |
| Lum B | 28 | (16.6) | 9 | (17.6) | 0.86 |

TABLE 3
Patient characteristics of dataset #2

| | | | |
|---|---|---|---|
| Characteristic | CA N = 85(%) | AA N = 44 (%) | p value |

| | | | | | |
|---|---|---|---|---|---|
| Age | | | | | |
| <40 | 9 | (10.6) | 5 | (11.4) | |
| 40-49 | 20 | (23.5) | 11 | (25) | |
| >=50 | 54 | (63.5) | 26 | (59.1) | |
| Missing | 2 | (2.4) | 2 | (4.5) | 0.90 |
| Node Status | | | | | |
| Positive | 32 | (37.6) | 24 | (54.6) | |
| Negative | 51 | (60) | 18 | (40.9) | |
| Unknown | 2 | (2.4) | 2 | (4.5) | 0.11 |
| Size | | | | | |
| 1 | 23 | (27.1) | 9 | (20.5) | |
| 2 | 41 | (48.2) | 20 | (45.5) | |
| +3 | 19 | (22.4) | 12 | (27.2) | |
| Missing | 2 | (2.3) | 3 | (6.8) | 0.50 |
| ER status | | | | | |
| Positive | 57 | (67.1) | 23 | (52.3) | |
| Negative | 25 | (29.4) | 19 | (43.2) | |
| Missing | 3 | (3.5) | 2 | (4.5) | 0.26 |
| PR status | | | | | |
| Positive | 41 | (48.2) | 19 | (43.2) | |
| Negative | 34 | (68) | 21 | (47.7) | |
| Missing | 10 | (11.8) | 4 | (9.1) | 0.69 |
| Her2 Status | | | | | |
| Positive | 21 | (24.7) | 11 | (25) | |
| Negative | 58 | (68.2) | 30 | (68.2) | |
| Missing | 6 | (7.1) | 3 | (6.8) | 1.00 |

TABLE 4
Characteristics of TCGA dataset

| | | | |
|---|---|---|---|
| Characteristic | CA N = 449(%) | AA N = 49 (%) | P value |

| | | | | |
|---|---|---|---|---|
| Age | | | | |
| <40 | 27 | (6) | 7 | (14.3) |
| 40-49 | 89 | (19.8) | 11 | (22.4) |
| >=50 | 333 | (74.2) | 30 | (61.2) |

| | | | | |
|---|---|---|---|---|
| Missing | 0 | 1 | (2) | .002 |

| Node Status | | | | | |
|---|---|---|---|---|---|
| 0 | 2 | (.4) | 0 | | |
| 1 | 44 | (9.8) | 4 | (8.2) | |
| 2 | 55 | (12.2) | 5 | (10.2) | |
| +3 | 341 | (75.9) | 37 | (75.5) | |
| Missing | 7 | (1.6) | 3 | (6.1) | 0.28 |
| ER status | | | | | |
| Positive | 449 | (100) | 49 | (100) | |
| PR status | | | | | |
| Positive | 384 | (85.5) | 40 | (81.6) | |
| Negative | 62 | (13.8) | 9 | (18.4) | |
| Missing | 3 | (.7) | 0 | | 0.59 |
| HER2 Status | | | | | |
| Positive | 61 | (13.6) | 3 | (6.1) | |
| Negative | 240 | (53.5) | 19 | (38.8) | |
| Equivocal | 83 | (18.5) | 15 | (30.6) | |
| Missing | 65 | (14.5) | 12 | (24.5) | 0.02 |

Normal datasets: The normal breast tissue dataset (GSE43973), had microarray data from 12 AA women and 98 CA women. The tumor adjacent normal tissue dataset (GSE50939) had microarray data from 14 AA patients and 52 CA patients.

Enrichment analysis: For RNA analysis, p-values were obtained by comparing each gene between AA and CA tumors using Wilcoxon Rank Sum test. These p-values were rank-ordered and q-values were computed. All genes with q<0.25 (p<0.1) were considered candidates in each dataset. Candidate genes with RNA dysregulation in the same direction across two datasets were curated to form the final list of genes for downstream analyses. For overall patterns of RNA dysregulation in the union of the three datasets, each gene was included only once if it was dysregulated in the same direction in multiple datasets but included twice if it was dysregulated in opposite directions in multiple datasets. Fisher's exact test determined p-values for overall patterns of up and down-regulation.

For mutation analysis, any gene with 2% incidence (i.e. >1 mutation in AA tumors) was considered. All non-synonymous mutations were included irrespective of category (i.e. missense, nonsense, frameshift, etc) or predicted pathogenicity. Expected rates of mutation frequency were calculated based on total number of mutations identified in the entire patient population and compared to observed rates in AA and CA tumors respectively. Fisher's exact test determined p-values by comparing observed to expected frequency of each mutation.

For functional pathway analysis, functional category was determined based on literature searches for each gene of each pathway used in this targeted analysis (Table 1). Each gene was then assigned to sensor, scaffold, DNA repair and downstream effector categories based on a literature search. If a gene fell into two functional categories, it was considered in statistical analyses of each category in turn. The number of candidate genes in each category was compared to the total number of genes in that category using a Fisher's exact test.

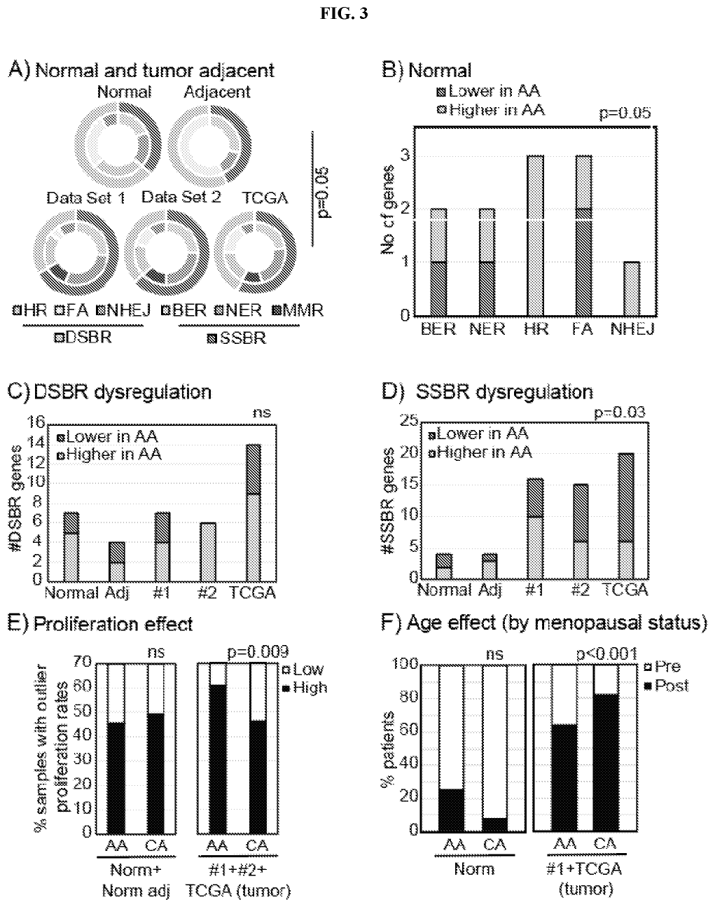

For proliferation analyses in FIG. 3, MKI67 (gene name for Ki67) RNA levels were used and the top 20% and bottom 20% of MKI67 expressing tumors were considered as “High” and “Low” proliferators respectively. Fisher's exact test determined p-values.

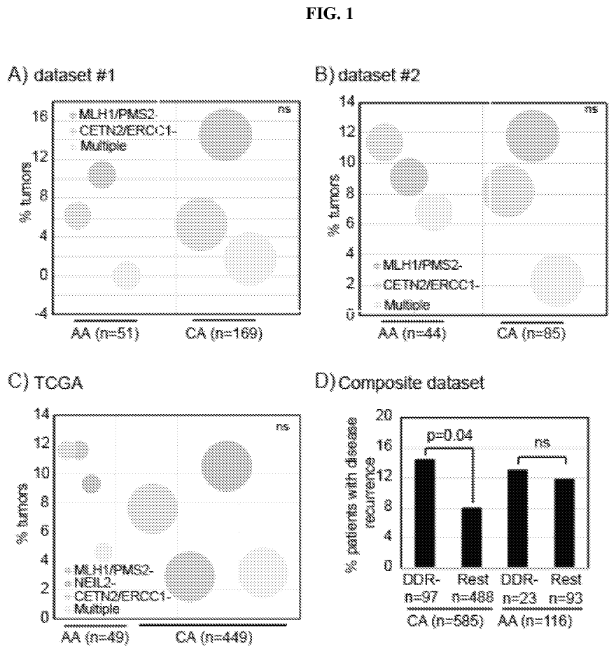

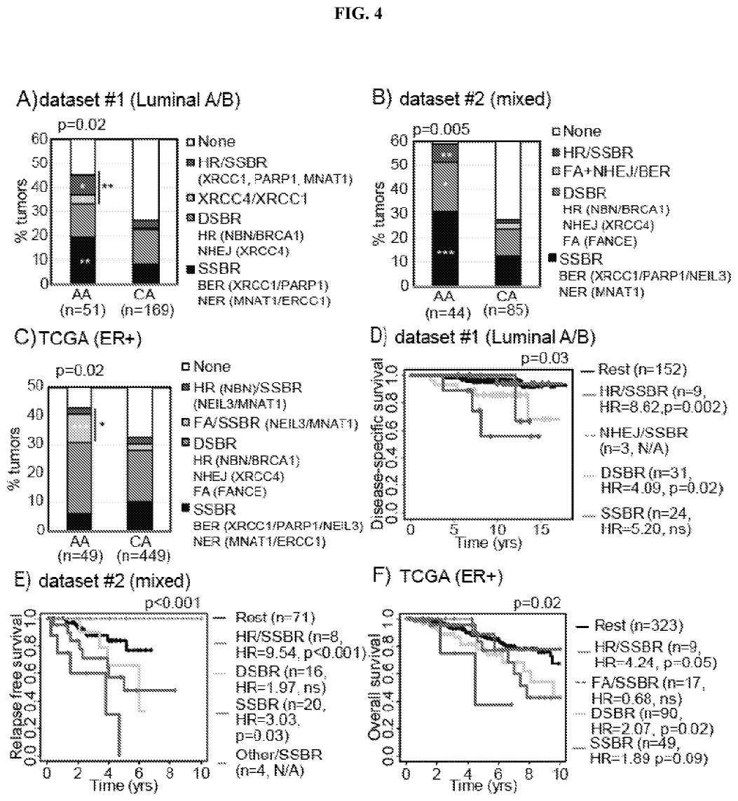

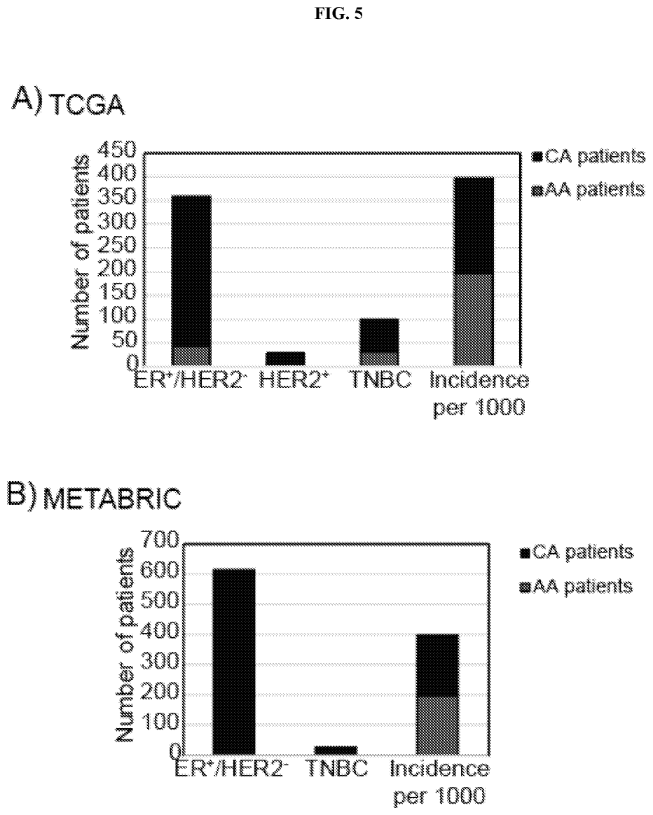

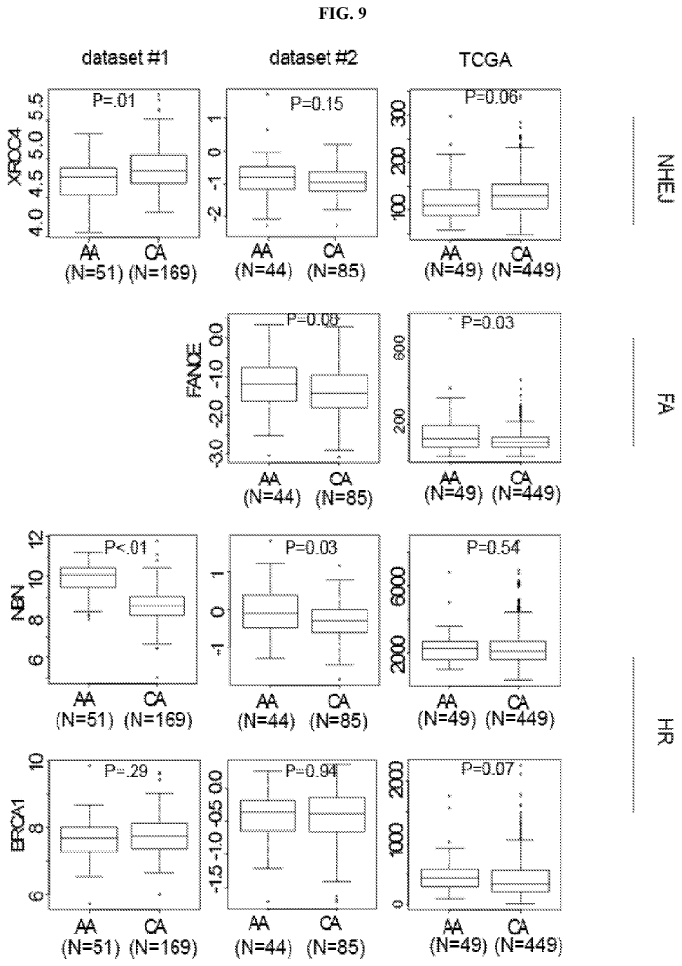

Survival analysis: For univariate and multivariate analyses, all tumors with associated survival data in each dataset were used, with restriction to luminal A/B tumors in dataset #1 and ER+ tumors in TCGA. Outcome measures used were disease-free survival for dataset #1 and TCGA, and recurrence-free survival in dataset #2 in FIG. 1, and in FIGS. 4 and 9, disease-specific survival for dataset #1, recurrence-free survival for dataset #2 and overall survival for TCGA. These different outcome measures were used because they had the largest sample size associated with them in each dataset. Factors included in multivariate analyses were PAM50 status and tumor stage for dataset #1, PAM50, tumor size and nodal status for dataset #2 and PR/HER2 status and tumor stage for TCGA. For all datasets, race was included as a categorical, and age as a continuous, variable. Only samples with survival metadata were included in the analysis.

Statistical analysis: Missing data were imputed with “NA” from mutation, expression, and survival data analysis. Samples classifying for more than one category (e.g. SSBR and DSBR dysregulation) were treated as separate set for statistical comparisons. Two-tailed Wilcoxon rank sum tests were used for two-sample tests of comparing continuous data and Pearson's Chi Square test (or Fisher's Exact test) was used for comparing categorical data. Log rank test calculated p-values for survival analyses and Cox regression determined proportional hazards.

### Results

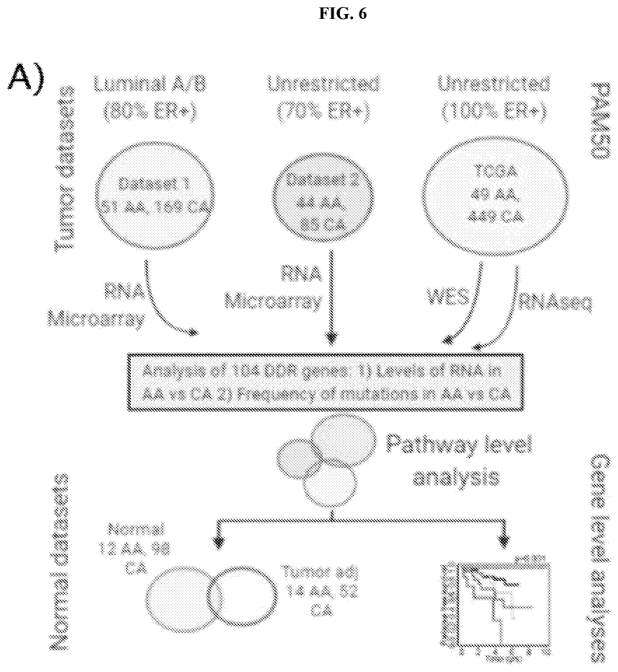

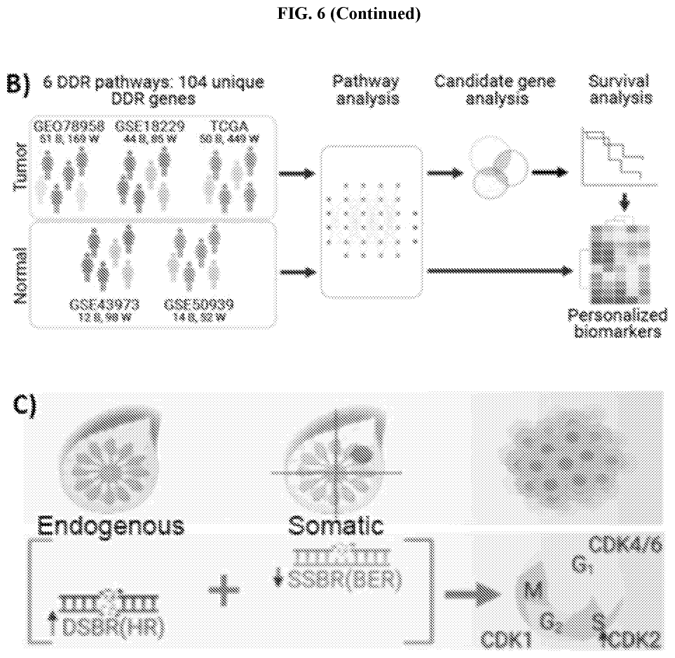

Defects in previously identified DNA repair genes do not associate with outcome in African American patients. The frequency with which DDR genes known to induce endocrine therapy resistance when lost (MMR: MLH1 and PMS2, NER: CETN2 and ERCC1, and BER:NEIL2) are downregulated in AA tumors was first investigated from three selected datasets (FIG. 6). In all three datasets (Tables 2-4), frequency of downregulation of RNA levels of these DDR genes was similar between AA and CA tumors with no demonstrable enrichment by race (FIGS. 1A-C). Whether downregulation of these DDR genes associated with poor disease-free survival in AA patients was next tested, as it did in CAs. Because of the small sample size of AA patients in each dataset, data from all three datasets for this analysis were combined. There was no significant difference in disease recurrence between AA patients whose tumors had downregulation of these DDR genes vs patients whose tumors did not downregulate these genes (14% vs 12% disease recurrence, FIG. 1D). In contrast, and as expected, a statistically significant increase was observed in disease recurrence in CAs with 14% of patients whose tumors downregulated these DDR genes having disease recurrence compared to only 8% of CAs (p=0.04) whose tumors did not detectably downregulate these DDR genes (FIG. 1D). Overall, this analysis identified no enrichment in AA ER+ tumors for downregulation of the four DDR genes previously identified as driving poor outcome in CAs.

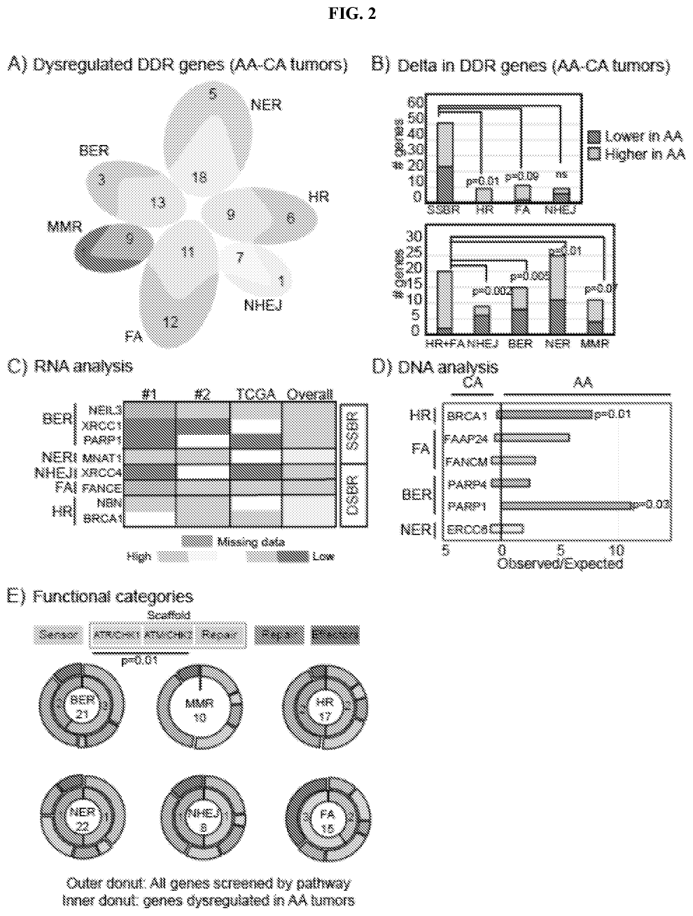

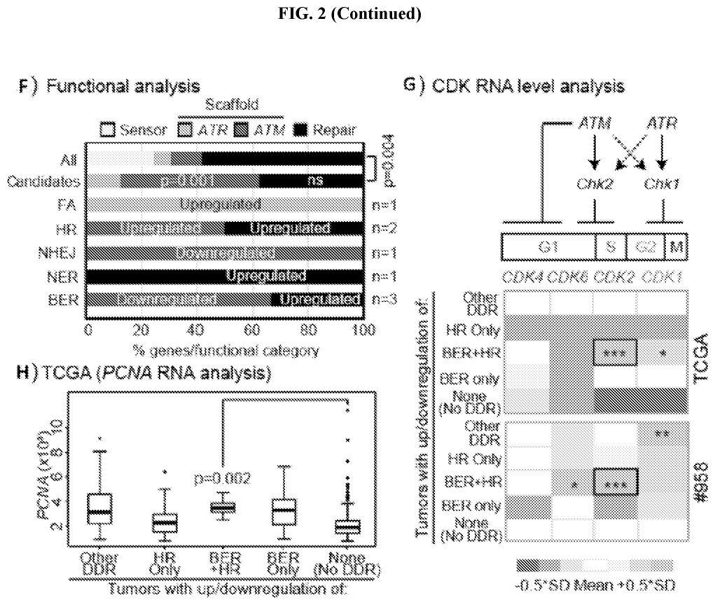

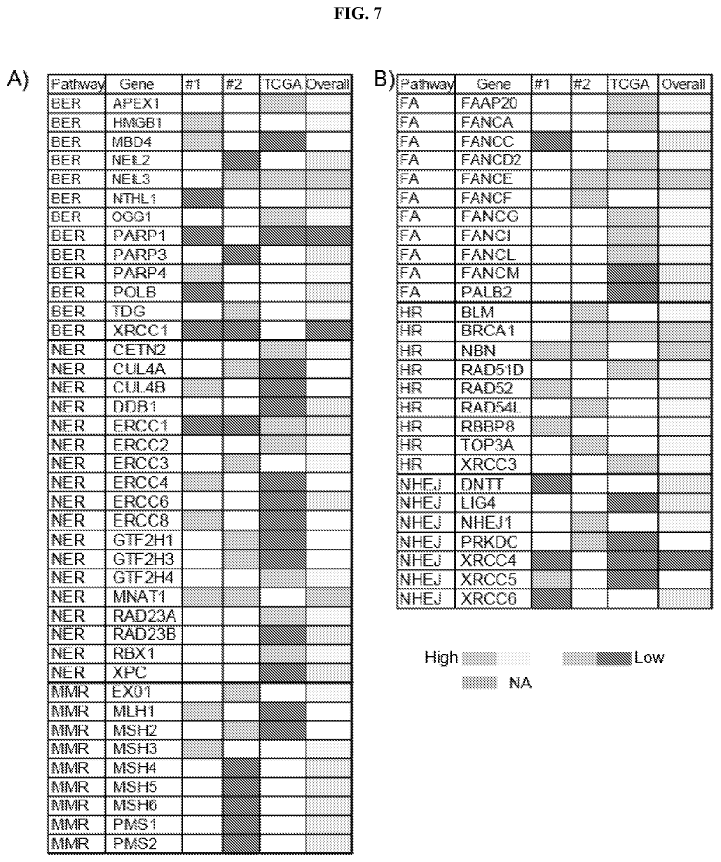

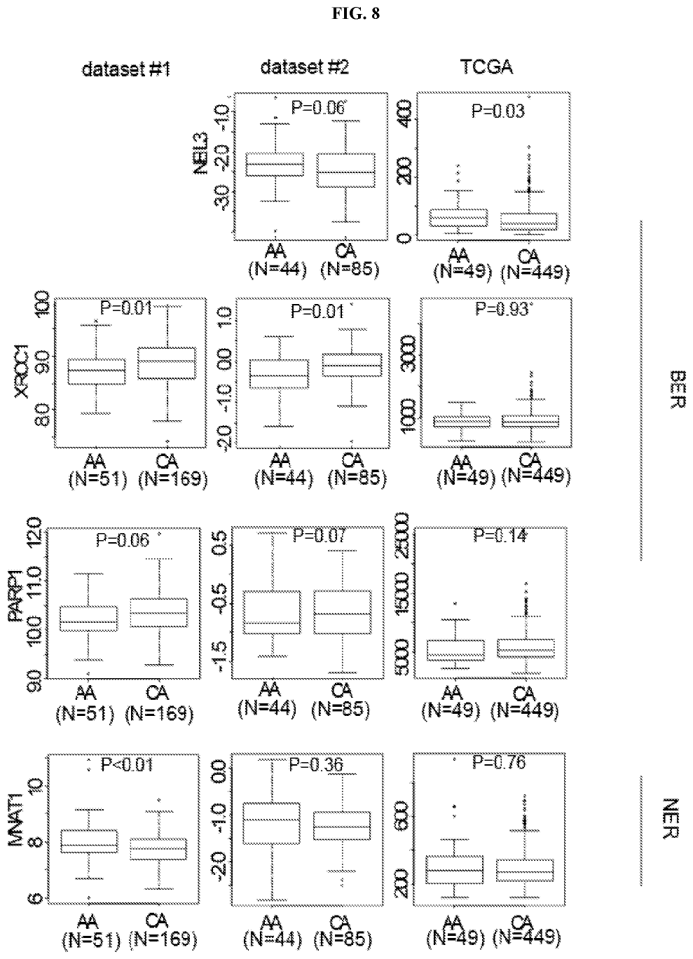

Landscape of DNA repair dysregulation in African American tumors was studied. To identify whether AA tumors have a distinct pattern of DDR dysregulation, RNA levels of 104 DDR genes from six principle DDR pathways (Table 1) were compared between AA and CA tumors in each of three independent datasets: dataset #1 (Table 2), dataset #2 (Table 3) and TCGA (Table 4). From the union of the three datasets, 67 genes were either up- or downregulated in AA tumors relative to CAs (q<0.25, FIG. 7). SSBR genes were enriched in this gene set with 80% of NER genes and 90% of BER genes being either up- or down-regulated in AA tumors relative to CAs in at least one dataset (FIG. 2A). Fanconi Anemia (FA) genes were similarly heavily represented (90% of genes making the cut-off). The remaining pathways had 50-60% of their genes represented (FIG. 2A). Overall, SSBR genes (NER, BER and MMR) had lower RNA levels in AA tumors relative to CAs, while DSBR genes (FA and HR) had higher RNA levels (FIG. 2B). Non-homologous end joining (NHEJ) was the only DSBR pathway where gene expression was preferentially downregulated in AA tumors (FIG. 2B). The list of genes dysregulated in the same direction were identified in at least two of the three datasets analyzed. This gene list consisted of three BER genes (NEIL3, XRCC1, PARP1), one NER gene (MNAT1), one NHEJ gene (XRCC4), one FA gene (FANCE) and two HR genes (NBN and BRCA1) (FIG. 2C). Consistent with overall patterns of dysregulation, FA and HR (DSBR) genes were upregulated, XRCC4 (NHEJ) was downregulated, and two of the three BER genes were downregulated (FIG. 2C, FIG. 8, and FIG. 9).

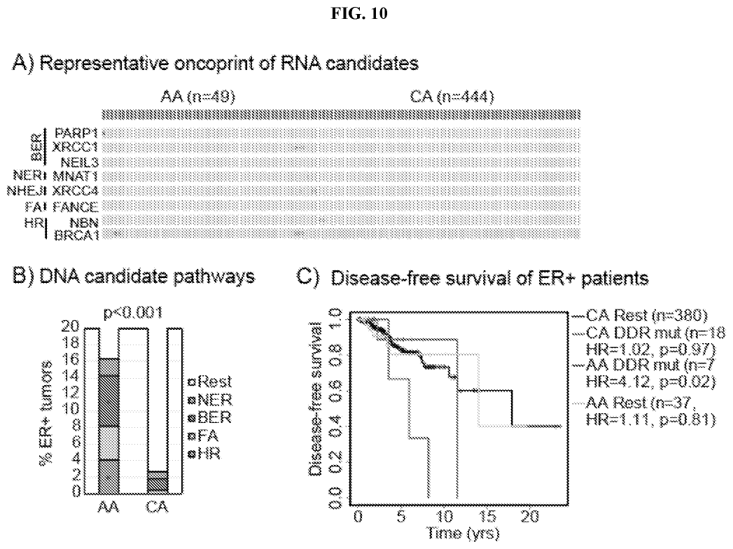

Using whole exome sequence data from TCGA, whether any DDR genes were enriched for mutations in AA tumors was next tested. Other than PARP1, none of the DDR genes differentially regulated in AA tumors from the RNA analysis were mutated in AA tumors (FIG. 10A). Overall, 16% of AA tumors had mutations in at least one DDR gene, compared to only 3% of CA tumors (p<0.001), with specific enrichment for mutations in genes from BER and HR pathways (FIG. 10B). In total, six genes: ERCC6 (NER), PARP1 and PARP4 (BER), FANCM and FAAP24 (FA) and BRCA1 (HR) had increased mutational frequency in AA tumors vs CAs, of which, PARP1 and BRCA1 (also identified in RNA analysis) reached statistical significance (p=0.01, p=0.03) respectively (FIG. 2D). Mutations in any DDR gene associated with significantly worse disease-free survival in AA patients (HR=4.12, p=0.02, FIG. 10C).

To test whether any functional patterns emerged in the DDR genes identified as differentially regulated at RNA level/mutated in AA tumors, distribution of these genes was assessed in the cascade of events involved in each DDR pathway. Genes were categorized based on their primary function as Sensor, Scaffold, Repair or Effectors (Table 1). Sensors sense the presence of specific types of DNA damage, Scaffold proteins serve to stabilize and activate other proteins at the site of damaged DNA, while Repair and Effector proteins are directly involved in repairing damaged DNA. Scaffold proteins were further categorized as cell cycle checkpoint kinase scaffolds that stabilize and activate ATM/Chk2 and ATR/Chk1 kinases or repair scaffolds that stabilize and activate downstream DNA repair proteins. Components of any DDR pathway predominantly function as sensor or repair/effector proteins, with only 15-20% of the pathway proteins identified as cell cycle checkpoint scaffolds (FIG. 2E). However, the majority of DDR genes dysregulated in AA tumors are ATM/ATR scaffolds. This is in keeping with previous findings that DDR genes contribute to endocrine therapy resistance by dysregulating cell cycle checkpoint activation in response to endocrine therapy. Similarly presented in FIG. 2F, components of all DDR pathways predominantly function as Sensor or Repair proteins, with only 15% of proteins in any DDR pathway constituting a Scaffold. However, ˜60% of DDR genes differently expressed in tumors from Black breast cancer patients are ATM/ATR Scaffolds, a significant enrichment for association with cell cycle regulation.

To test this association further, RNA levels were analyzed for each of four principle cyclin-dependent kinases (CDKs): CDK1, CDK2 and CDK4/6 in tumors with up- or down-regulation of the 8 shortlisted DDR genes. CDK RNA levels were able to be assessed specifically in relation to these pathways since a high proportion of tumors demonstrated differential expression of HR or BER genes. Tumors with differential expression of genes from any of the other DDR pathways were grouped together as there were not sufficient tumors with differential expression of genes from each individual pathway to constitute an independent group. A subset of tumors had coincident up- and down-regulation of HR and BER genes respectively, referred to as HR/BER tumors. This subset significantly upregulated CDK2, a positive regulator of the S phase of the cell cycle, relative to all other subsets across datasets (FIG. 3G). Additionally, in TCGA, HR/BER tumors significantly upregulated PCNA, another marker of S phase progression (FIG. 3H). When comparing CDK gene expression between Black and white patient tumors independent of DDR gene expression, significant upregulation of CDK1, CDK4 and CDK6 in tumors from Black patients (p=8.05e-05, p=0.0001 and p=0.004, respectively) was found, but no difference in CDK2. These data suggest a new association between S phase progression and the DDR landscape of ER+ tumors from Black women.

Persistent upregulation of double strand break repair genes in normal breast tissue from African American women was observed. To understand whether differences in DDR dysregulation patterns in AA tumors are driven by endogenous differences in gene expression of breast tissue, microarray gene expression data were used from two publicly available datasets: GSE43973 with samples from reduction mammoplasty (12 AA and 98 CA women) and GSE50939 with samples from tumor-adjacent normal tissue (14 AA and 67 CA patients) (Pirone et al., Age-associated gene expression in normal breast tissue mirrors qualitative age-at-incidence patterns for breast cancer, Cancer Epidemiol Prev Biomark 21(10):1735-1744, 2012; Casbas-Hernandez et al., Tumor intrinsic subtype is reflected in cancer-adjacent tissue, Cancer Epidemiol Prev Biomark 24(2):406-414, 2015). Overall, the predominant difference in DDR gene expression between AA and CA breast tissue was upregulation of DSBR genes, while SSBR genes were rarely different between these two cohorts (FIGS. 3A-3D). Even NHEJ genes which are preferentially downregulated in AA tumors were upregulated in the normal breast (FIG. 3B). The number of dysregulated genes in the normal breast of AAs vs CAs was far fewer than that seen in tumors (FIGS. 3B-3D).

Whether baseline upregulation of DSBR genes in AA normal breast tissue and preferential SSBR downregulation in tumors associates with higher proliferation in AA tumors or younger age of AA women in the datasets was next tested. Both proliferation and age are known to affect DDR gene expression and AA breast cancer patients present with more proliferative tumors at a younger age (Comparison of breast cancer molecular features and survival by African and European ancestry in The Cancer Genome Atlas, Breast Cancer, JAMA Oncology, JAMA Network). In combined data from normal and tumor adjacent normal datasets, using gene expression of MKI67 as a proliferative index, no differences between AA and CA women were found (45% vs 49% were highly proliferative, FIG. 3E). However, in the combined tumor datasets, proliferation index was significantly higher in AA tumors (61% of tumors) compared to CAs (46%) p=0.009 (FIG. 3E). Therefore, it is possible that the loss of SSBR gene expression in AA tumors is associated with higher proliferation. A similar trend was detected when age was considered using menopausal status as a surrogate. While no statistically significant difference was found in age in women represented in normal datasets, the number of post-menopausal tumor samples was significantly higher in CA patients (81%) compared to AAs (63%), p<0.001 (FIG. 3F). Neither of these parameters explains the higher DSBR gene expression observed in normal AA breast tissue.

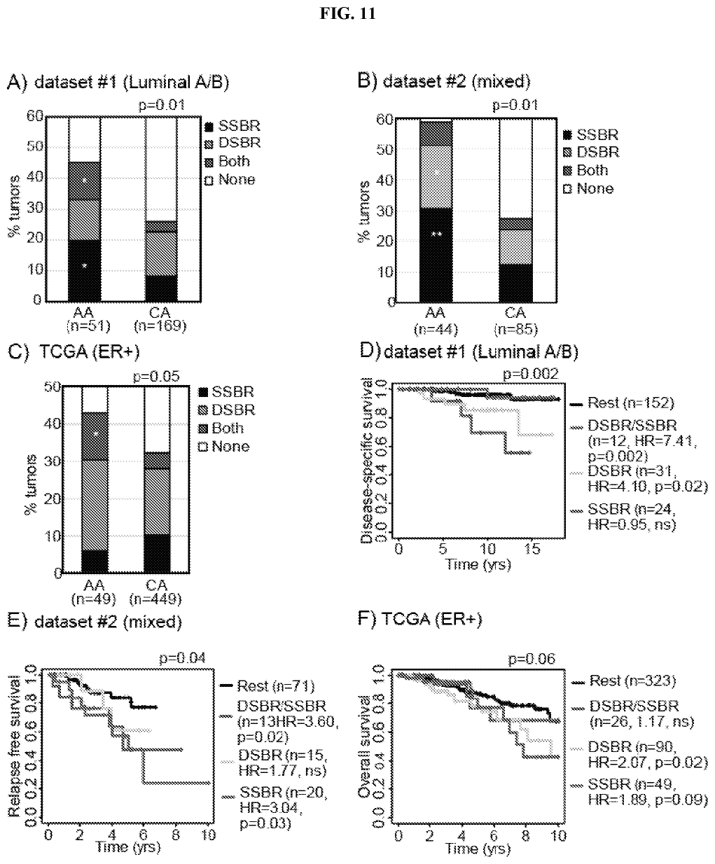

African American-specific DNA repair dysregulation associates with poor patient outcomes. To assess prevalence of DDR dysregulation in AA patients, the proportion of tumors with dysregulation of any of our DDR genes was tested (DSBR: HR—upregulation of NBN, BRCA1, FA—upregulation of FANCE, NHEJ—downregulation of XRCC4; SSBR: BER—downregulation of PARP1, XRCC1, upregulation of NEIL3, NER—upregulation of MNAT1) in AAs vs CAs. In datasets #1 and #2, AA tumors dysregulated at least one SSBR gene with a >2-fold increase in frequency relative to CAs (p=0.01 in each case, FIGS. 11A-B). In dataset #2 alone, AA tumors also dysregulated at least one DSBR gene with significantly increased frequency relative to CAs (FIG. 11B). In TCGA, frequency of SSBR and DSBR gene dysregulation was comparable between AA and CA tumors (FIG. 11C). However, in all three datasets, a subset of tumors that were significantly enriched in AA patients was observed. This subset simultaneously dysregulated SSBR and DSBR genes, and accounted for >10% of AA tumors, but <3% of CA tumors, >3-fold enrichment (FIG. 11A-11C). This subset also associated with significantly worse outcome in datasets #1, #2 and TCGA, irrespective of whether patients were AA or CA (FIGS. 11D-11F).

To further understand this subset, individual gene dysregulation was parsed. In all three datasets, simultaneous dysregulation of one of the two HR genes was observed, NBN or BRCA1, with different SSBR genes (an HR/SSBR subset). The NER pathway gene, MNAT1 was implicated in this simultaneous dysregulation in all three datasets (FIGS. 4A-4C). In datasets #1 and #2, the HR/SSBR subset was significantly enriched in AAs (6-8%) relative to CAs (1-3%) (FIG. 4A-4B), while in TCGA, an FA/SSBR subset was significantly enriched (10% vs 2%, p<0.001), FIG. 4C). This is likely because of all three datasets, TCGA had the best representation of FA genes (which were largely missing from datasets #1 and #2), and the most frequent upregulation of FANCE in AA tumors.

Since the HR/SSBR subset was represented in all datasets analyzed and was significantly enriched in AA tumors in two of three datasets, its association with outcomes was next tested. Each dataset was broken up into patients whose tumors dysregulated any SSBR gene (downregulation of XRCC1/XRCC4/PARP1, upregulation of NEIL3/MNAT1) alone and any DSBR gene (upregulation of NBN/BRCA1/FANCE) alone. Tumors with simultaneous dysregulation of SSBR and DSBR genes were divided into those with simultaneous upregulation of either HR gene, NBN/BRCA1, with dysregulation of any SSBR gene (downregulation of XRCC1/PARP1, upregulation of NEIL3/MNAT1). Associations with survival outcome of each of these groups were compared relative to patients whose tumors had no detectable dysregulation of any of these genes. The only patient group that consistently and significantly associated with worse disease-specific survival (dataset #1, HR=8.62, p=0.002, FIG. 4D), relapse-free survival (dataset #2, HR=9.54, p<0.001, FIG. 4E) and overall survival (TCGA, HR=4.24, p=0.05, FIG. 4F) was the HR/SSBR subset. On the other hand, the simultaneous dysregulation of non-HR DSBR candidates with any SSBR gene did not associate with survival outcomes in any dataset (FIGS. 4D-4F).

Next, Cox Proportional Hazards assessment was conducted to understand the effect of confounding factors (tumor stage or tumor grade/nodal status, PAM50 or PR/HER2 status, age and race) on associations between DDR dysregulation and survival. In datasets #1 and #2, but not in TCGA, association of HR/SSBR tumors was found with poor survival remained significant even in a proportional hazards assessment (Tables 5-7). Age and race did not remain significant in any of the three datasets, suggesting that the unique candidate gene set identified by analyzing AA patient tumors is likely a poor prognostic factor regardless of race, although significantly enriched in AAs.

To summarize, to investigate whether ER+ tumors from Black patients have a distinct pattern of DDR regulation that contributes to poor outcomes, RNA levels of 104 DDR genes from six principle DDR pathways were assessed in each of three datasets described in Tables 1-3 (see FIGS. 6A-6B). Together, the above results identify specific patterns of double strand break repair upregulation in normal breast and single strand break repair up- or down-regulation in ER+ tumor tissue from Black women that are distinct from that seen in white women. Moreover, the DNA repair landscape uncovered in tumors from Black women associates with increased CDK2 gene expression and worse outcome by every disease measure analyzed across three independent datasets (see FIG. 6C).

DNA repair proteins are natural molecular conduits between external stimuli and cellular response. Exposure to genotoxins or hypoxia, for instance, can induce a cell to up- or downregulate its DDR signaling. Not only do DDR proteins repair damaged DNA they also activate cell cycle checkpoints and engage apoptotic pathways. Therefore, DDR pathways assist in understanding how molecular factors that translate environmental stimuli into cellular phenotypes may be altered by race/ethnicity.

TABLE 5
Cox proportional hazards for dataset #1

| Factor | HR | CI | p | n |
|---|---|---|---|---|
| DDR status | | | | |
| Rest | — | | | 152 |
| DSBR | 2.02 | 0.53-7.61 | 0.30 | 31 |
| SSBR | 0.82 | 0.08-8.06 | 0.86 | 24 |
| HR/SSBR* | 6.44 | 1.32-31.31 | 0.02 | 9 |
| Other/SSBR | 1.82 | 0.16-20.88 | 0.63 | 3 |
| PAM50 | | | | |
| LumA | — | | | 182 |
| LumB | 0.92 | 0.23-3.67 | 0.90 | 37 |
| Tumor stage | | | | |
| i | — | | | 97 |
| ii | 1.42 | 0.30-6.63 | 0.66 | 95 |
| iii+* | 11.9 | 2.77-50.73 | <0.001 | 26 |
| Age | 1.01 | 0.97-1.05 | 0.671 | 218 |
| Race | | | | |
| AA | — | | | 51 |
| CA | 0.78 | 0.22-2.78 | 0.71 | 168 |

TABLE 6
Cox proportional hazards for dataset #2

| Factor | HR | CI | p | n |
|---|---|---|---|---|
| DDR status | | | | |
| Rest | — | | | 71 |
| DSBR | 0.83 | 0.19-2.92 | 0.51 | 16 |
| SSBR* | 4.38 | 1.25-16.85 | 0.03 | 19 |
| HR/SSBR*** | 16.84 | 12.92-73.13 | <.001 | 8 |
| PAM50 | | | | |
| LumA | — | | | 50 |
| LumB′ | 5.21 | 1.25-33.21 | 0.05 | 28 |
| HER2*** | 22.41 | 3.47-133.89 | <.001 | 15 |
| Basal-like*** | 40.42 | 7.58-225.46 | <.001 | 17 |
| Claudin-low | 1.9 | 0.35-47.05 | 0.64 | 7 |
| Normal-like*** | 180.1 | 8.90-3839.05 | <.001 | 2 |
| Tumor size | | | | |
| 1 | — | | | 32 |
| 2 | NA | N/A | N/A | 57 |
| 3 | N/A | N/A | N/A | 28 |
| Unk | N/A | N/A | N/A | 2 |
| Node | 0.73 | 0.24-2.22 | 0.58 | 119 |
| Age | 1 | 0.96-1.04 | 0.88 | 119 |
| Race | | | | |
| AA | — | | | 40 |
| CA | 0.92 | 0.33-2.54 | 0.87 | 79 |

TABLE 7
Cox proportional hazards for TCGA

| Factor | HR | CI | p | n |
|---|---|---|---|---|
| DDR status | | | | |
| Rest | — | | | 328 |
| DSBR* | 2.1 | 1.05-4.22 | 0.04 | 90 |
| SSBR | 1.68 | 0.05-3.25 | 0.22 | 49 |
| HR/SSBR | 1.77 | 0.22-14.12 | 0.59 | 9 |
| Other/SSBR | 0.41 | 0.05-3.25 | 0.40 | 17 |
| PR status | | | | |
| Neg | — | | | 71 |
| Pos | 0.72 | 0.33-1.58 | 0.99 | 419 |
| HER2 status | | | | |
| Neg | — | | | 345 |
| Pos* | 2.09 | 1.11-3.95 | 0.02 | 110 |
| Tumor Stage | | | | |
| 1 | — | | | 153 |
| 2 | 0.88 | 0.44-1.79 | 0.73 | 257 |
| 3 | 1.43 | 0.67-3.04 | 0.35 | 83 |
| Age | 1.02 | 0.99-1.04 | 0.15 | 492 |
| Race | | | | |
| AA | — | | | 49 |
| CA | 1.91 | 0.45-8.16 | 0.38 | 444 |

### Example 2. The Impact of DNA Damage Repair Genes on Poor Outcome in African American Breast Cancer Patients

African American (AA) breast cancer patients have worse outcomes than Caucasian Americans (CAs). DNA damage repair (DDR) genes drive poor outcome in CA estrogen receptor (ER)+ breast cancer patients. Whether DDR genes similarly impact survival in AAs is unknown. Identifying AA-specific patterns of DDR dysregulation could change how predictive/prognostic biomarkers are tailored. DDR dysregulation in ER+ AA patient tumors was characterized and associations with clinical outcome was tested by analyzing three independent tumor and two normal breast datasets. Tumor datasets: (1) GSE78958 (2) GSE18229 (3) The Cancer Genome Atlas (TCGA). Normal datasets: (4) GSE43973 (5) GSE50939.

Up/down-regulation of 104 DDR genes was assessed in AA samples vs CAs. Survival associations were assessed for genes dysregulated in multiple datasets. Overall, RNA levels of single strand break repair (SSBR) genes were downregulated in AA tumors and double strand break repair (DSBR) genes were upregulated compared to CAs. While SSBR downregulation was mainly detected in tumors, DSBR upregulation was detectable in both tumor and normal breast AA samples. Seven specific DDR genes identified as dysregulated in AAs vs CAs in multiple datasets associated with poor survival. A subset of tumors with simultaneous dysregulation of homologous recombination and single strand break repair genes was enriched in AAs and had associated consistently with poor survival.

Overall, these results constitute the first systematic analysis of differences in DDR regulation in AA ER⁺ tumors and normal tissue vs CAs. A profile of DDR dysregulation enriched in AA patients, which associates with poor outcome, was identified. These results suggest a distinct molecular mechanism of DDR regulation in AAs to allow biomarker profiles by race and improved precision medicine for underserved populations.

### Example 3. Distinct Pattern of DNA Damage Repair Dysregulation in ER⁺/HER2⁻ Breast Tumors in African American Women

In this cohort study a distinct DNA damage repair dysregulation was detected in African American women with ER⁺/HER2⁻ breast cancer. Molecular differences in RNA levels and mutational frequency were observed between African American and Caucasian tumors. Most strikingly, simultaneous downregulation of two DNA repair pathways was enriched in African American patients and associated with poor survival.

Disparity in breast cancer outcome between African American and Caucasian women is well established. Described herein is a map of the DNA repair landscape in ER⁺/HER2⁻ breast cancer from African American women and a significant association with worse survival outcome.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A method of prognosing and/or diagnosing an estrogen receptor positive (ER⁺) breast cancer in an African American subject in need thereof, comprising
a. measuring a level of expression of one or more DNA damage repair (DDR) genes in a sample from the African American subject, wherein the one or more DDR genes is selected from the group consisting of XPC, XPA, RBX1, RAD23B, RAD23A, MNAT1, GTF2H5, GTF2H4, GTF2H3, GTF2H2, GTF2H1, ERCC8, ERCC6, ERCC5, ERCC4, ERCC3, ERCC2, ERCC1, DDB2, DDB1, CUL4B, CUL4A, CETN2, XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, APEX1, PMS2P3, PMS2, PMS1, MSH6, MSH5, MSH4, MSH3, MSH2, MLH3, MLH1, EXO1, XRCC6, XRCC5, XRCC4, PRKDC, POLM, NHEJ1, LIG4, DNTT, XRCC3, XRCC2, TOP3B, TOP3A, SHFM1, RBBP8, RAD54L, RAD54B, RAD52, RAD51D, RAD51B, RAD51, NBN, MUS81, SLX1B, SLX1A, GEN1, EME2, EME1, DMC1, BRCA1, BLM, SLX4, PALB2, FANCM, FANCL, FANCI, FANCG, FANCF, FANCE, FANCD2, FANC, FANCB, FANCA, FAAP24, FAAP20, and BRIP1; and
b. comparing the level of expression of one or more DNA damage repair (DDR) genes to a control level of expression to obtain a comparison expression value,
thereby prognosing and/or diagnosing the ER⁺ breast cancer in the African American subject in need thereof.

2-14. (canceled)

15. A method of prognosing and/or diagnosing an estrogen receptor positive (ER⁺) breast cancer in an African American subject in need thereof, comprising
a. measuring a level of expression of one or more of NBN, BRCA1, FANCE, MNAT1, NEIL3, XRCC4, XRCC1, and PARP1 in a sample from the African American subject; and
b. comparing the level of expression of one or more of NBN, BRCA1, FANCE, MNAT1, NEIL3, XRCC4, XRCC1, and PARP1 to a control level of expression to obtain a comparison expression value,
thereby prognosing and/or diagnosing the ER⁺ breast cancer in the African American subject in need thereof.

16-47. (canceled)

48. A method of treating an estrogen receptor positive (ER⁺) breast cancer in a susceptible African American subject, comprising
a. identifying the susceptible African American subject by
i. measuring a level of expression of one or more DNA damage repair (DDR) genes involved in a homologous recombination (HR) pathway, wherein the one or more DDR genes involved in the HR pathway are selected from the group consisting of XRCC3, XRCC2, TOP3B, TOP3A, SHFM1, RBBP8, RAD54L, RAD54B, RAD52, RAD51D, RAD51B, RAD51, NBN, MUS81, SLX1B, SLX1A, GEN1, EME2, EME1, DMC1, BRCA1, and BLM; and a level of expression of one or more DDR genes involved in a base excision repair (BER) pathway, wherein the one or more DDR genes involved in the BER pathway are selected from the group consisting of XRCC1, UNG, TDG, SMUG1, POLB, PNKP, PARP4, PARP3, PARP2, PARP1, OGG1 NTHL1, NEIL3, NEIL2, NEIL1, MUTYH, MPG, MBD4, LIG3, HMGB1P4, HMGB1P1, HMGB1, APLF, APEX2, and APEX1; and
ii. comparing the level of expression of one or more DDR genes involved in the HR pathway and the level of expression of one or more DDR genes involved in the BER pathway to a control level of expression to obtain a comparison expression value; and
b. administering to the susceptible African American subject an effective amount of lone or more cyclin-dependent kinase 2 (CDK2) inhibitors; b) one or more proliferating cell nuclear antigen (PCNA) inhibitors; or c) one or more inhibitors of cyclin-dependent kinase 1 (CDK-1), CDK-4, and CDK-6, thereby treating the ER+ breast cancer in the African American subject.

49. The method of claim 48, wherein the level of expression of one or more DDR genes involved in the HR pathway is higher than the control level of expression, and the level of expression of one or more DDR genes involved in the BER pathway is lower than the control level of expression in the susceptible African American subject.

50. The method of claim 48, further comprising obtaining a sample from the African American subject in need thereof.

51. The method of claim 50, wherein the sample comprises biopsy, urine, feces, polyp, cerebrospinal fluid, blood, semen, nipple aspirate, or a combination thereof.

52. The method of claim 50, wherein the sample is obtained when the African American subject in need thereof is suspected of having breast cancer after breast test, mammogram, and/or breast ultrasound.

53. The method of claim 50, wherein the sample is obtained when the African American subject in need thereof experiences a recurrent or metastatic breast cancer after one or more treatments.

54. The method of claim 48, wherein the level of expression of one or more DDR genes involved in the HR pathway or the level of expression of one or more DDR genes involved in the BER pathway is measured by a method selected from the group consisting of quantitative PCR, Western blot, immunoblot, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, ligand binding assay, microarray, polyacrylamide gel electrophoresis such as SDS-PAGE, surface plasmon resonance (SPR), Forster resonance energy transfer (FRET), Bioluminescence resonance energy transfer (BRET), chemiluminescence, fluorescent polarization, phosphorescence, mass spectrometry, magnetic resonance imaging (MRI), or a combination thereof.

55. The method of claim 48, further comprising prognosing the African American subject in need thereof as later having poor disease-specific survival rate, relapse-free survival rate, and/or overall survival rate.

56. The method of claim 48, wherein the one or more CDK2 inhibitors comprise AT7519, AG-024322, Dinaciclib, Cyc065, Roniciclib, TG02, and/or Milciclib.

57. The method of claim 48, further comprising administering to the susceptible African American subject endocrine therapy, surgery, chemotherapy, and/or radiation.

58. The method of claim 57, wherein the endocrine therapy comprises one or more selective estrogen-receptor response modulators (SERMs), one or more aromatase inhibitors, one or more estrogen-receptor downregulators (ERDs), one or more Luteinizing hormone-releasing hormone agents (LHRHs), or a combination thereof.

59-64. (canceled)

65. The method of claim 54, wherein the level of expression of one or more DDR genes involved in the HR pathway or the level of expression one or more DDR genes involved in the BER pathway is measured by a method selected from the group consisting of quantitative PCR, enzyme-linked immunosorbant assay (ELISA), enzyme-linked immunospot (ELISPOT), radioimmunoas say (RIA), immunohistochemistry, immunoprecipitation, fluorescence activated cell sorting (FACS), microscopy, flow cytometry, microcytometry, protein binding assay, surface plasmon resonance (SPR), Förster resonance energy transfer (FRET), Bioluminescence resonance energy transfer (BRET), chemiluminescence, fluorescent polarization, phosphorescence, mass spectrometry, or a combination thereof.

66-69. (canceled)

70. The method of claim 48, wherein the one or more inhibitors of CDK-1, CDK-4, and CDK-6 comprise abemaciclib, Palbociclib, ribociclib, or a combination thereof.

71-75. (canceled)

76. The method of claim 48, wherein the sample comprises biopsy, urine, feces, polyp, cerebrospinal fluid, blood, or a combination thereof.

77-81. (canceled)

82. The method of claim 48, wherein the African American subject in need thereof has a stage I, II, III, or IV breast cancer.

83. The method of claim 48, wherein the African American subject in need thereof has a node status of 0, 1, 2, 3, or 3+.

84. The method of claim 48, wherein the African American subject in need thereof has a breast cancer that has tested positive or negative for progesterone receptors.

85. The method of claim 48, wherein the African American subject in need thereof has a breast cancer that has tested positive or negative for HER2 protein.
